(19) United States
(12) Patent Application Publication
(10) Pub. No.: US 2002/0099410 A1
(43) Pub. Date: Jul. 25, 2002

(54) Device and method for sealing a puncture in a blood vessel

(75) Inventor: Shlomo Ben David, Tel Aviv (IL)

Correspondence Address: OLIFF & BERRIDGE, PLC, P.O. BOX 19928, ALEXANDRIA, VA, 22320, US

(73) Assignee: BIO-SEAL TECH INC., Garfield, NJ

(21) Appl. No.: 09/946,592

(22) Filed: Sep. 6, 2001

Related U.S. Application Data
(63) Continuation-in-part of application No. 09/808,142, filed on Mar. 15, 2001.
Application No. 09/808,142 is a continuation-in-part of application No. 09/757,623, filed on Jan. 11, 2001.

(51) Int. Cl. A61B 17/08

(52) U.S. Cl. 606/213

(57) ABSTRACT

A sealing device for sealing a puncture in a blood vessel, the device being slidable over a guide tube and comprising a tubular housing, a resilient sealing member formed with a sealing portion spontaneously scalable upon deployment of the device into an operative state, an engaging portion for bearing against an external surface of the blood vessel and an anchoring assembly for engaging an internal surface of the blood vessel. The anchoring assembly comprises anchor members which are displaceable between a constricted position in which they blend with the tubular housing, and an operative position in which the anchors are laterally deployed to engage the internal surface of the blood vessel.

### RELATED APPLICATIONS

The present application is a continuation-in-part of application Serial No. 09/808,142 filed Mar. 15, 2001, which is a continuation-in-pat of application Serial No. 09757,623 filed Jan. 11, 2001.

### FIELD OF THE INVENTION

The present invention is generally in the field of homeostatic devices and in particular it is directed to a sealing device for sealing a puncture (incision) in a blood vessel of a patient during or after a. medical procedure. The invention is also concerned with a deploying member and a method for deploying the sealing device into the aperture's site.

### BACKGROUND OF THE INVENTION

During several surgical procedures, for example in treatment of vascular diseases, it is common practice to invade a blood vessel and introduce a treating or diagnostic device, e.g. balloons or various types of stents to operate on walls of the arteries, plaque removing devices, observation and flow diagnostic instruments, etc.

During such procedures, a blood vessel is punctured so as to allow introduction of the instrument through the artery and then maneuver it to the required site of operation. This is carried out in practice by introducing a guide tube often referred to as an “introducer sheath” a “guide sheath” or a “guide tube”, through which the instrument can then be easily maneuvered to the site of interest

A problem occurs once the procedure is complete and the guide tube has then to be removed, when the percutaneous puncture bleeds. Bleeding may result in hematoma or in severe cases to malfunction of critical organs and even death. Such bleeding is stopped, by a most common method, by simply applying pressure on to the puncture site by a medically trained person for a sufficiently long period of time until homeostasis takes place to spontaneously seal the puncture and stop the bleeding.

In cases of puncturing the femoral arteries, the required time may be as long as about 45 minutes or more and in some cases re-bleeding occurs if the patient is not in rest.

A variety of methods and devices have been suggested for replacing the traditional method disclosed above, some of which involve introducing chemical compounds which act as homeostasis catalysts or as adhering agents, whilst others aim at introducing various forms of plugging members into the puncture. The following is a list of prior art patents disclosing devices and methods for sealing punctured blood vessels: U.S. Pat. Nos. 4,705,040 4,890,612, 4,929,246, 5,108,420, 5,342,393, 5,350,399, 5,391,183, 5,613,974, 5,810,884, 5,861,003, 5,957,952, 5,984,950,6,007,563 and WO 98/31287.

It is an object of the present invention to provide novel and inventive devices for sealing a puncture or an incision formed in a blood vessel or in other body organs, as well as an associated deploying member for deploying the sealing member into a sealing position within the puncture. A further object of the invention is to provide a method utilizing the sealing member and the associated deploying assembly.

### SUMMARY OF THE INVENTION

The present invention calls for a sealing device and an associated deploying assembly, as well as a method for sealing a puncture or an incision formed in a body organ, typically but not limited in, a blood vessel.

By one feature of the present invention, the sealing device is introduced into the puncture over a guide tube (guide sheath) used for carrying out a medical procedure e.g. angioplasty, in which a probe is introduced into a blood vessel through the guide tube. The sealing member of the present invention is received within a deploying member used for displacing the sealing member into the puncture site and then deploying it into its operative-sealing position, in which it is anchored within the puncture and seals the puncture/incision. The sealing device is fitted with anchoring means to ensure suitable anchorage within the puncture and is useful for a variety of guide tubes on the one hand and for a variety of wall thicknesses of the blood vessel at the puncture site, on the other hand.

In accordance with an embodiment of the invention there is provided a sealing device for sealing a puncture in a blood vessel, the device being slidingly receivable over a guide tube and comprising a tubular, resilient sealing member formed with a sealing portion spontaneously sealable upon deployment of the device into an activated state, an engaging portion for bearing against an external surface of the blood vessel and a plurality of anchors fitted at their fore end with fasteners for engaging an internal surface of the blood vessel t; said anchors being displaceable between a constricted position in which they blend with the tubular sealing member, and an operative position in which the fasteners are laterally deployed to engage the internal surface of the blood vessel.

In accordance with one particular embodiment the anchors are fitted at their rear end with a manipulating bit engageable by a corresponding member of the deploying assembly.

In accordance with another embodiment, the sealing device comprises a plurality of anchors fitted at their fore end with a fastener which at the operative position spontaneously deforms into a laterally projecting position for engaging the internal surface of the blood vessel. For such spontaneous displacement the fasteners are pre-strained and retained at their constricted position by a suitable arresting arrangement (by a suitable sleeve member or a guide tube, prior to deploying) preventing them from spontaneous displacing into the operative position. In accordance with this embodiment; the fasteners are rearwardly facing thus adapted for engaging an internal surface of the blood vessel.

The present invention also suggests a unique application of a sealing device fitted with means for grabbing tissue of a blood vessel (or any other organ being sealed) surrounding the puncture (i.e. lip or edges of the puncture) and applying force in direction so as to adjoin edges of the puncture, thus reducing the section area of the puncture and speeding the sealing process.

In accordance with a particular embodiment of that application, the fasteners are fitted with spikes facing rearwards for engaging tissue of the blood vessel, wherein at the deployed state the anchors are biased radially inwardly, for constricting the size of the puncture in the blood vessel.

According to another embodiment of that application, there are provided several spears axially displaceable with respect to the resilient sealing member, said spears having a fore end fitted for grabbingly engaging the external tissue surface of the blood vessel at locations peripheral to the puncture. At least said fore end being displaceable radially inwardly so as to decease the imaginary delimited circle defined by the spears.

The radial inward force applied to the anchors and to the spears disclosed above is delivered by deformation of the resilient sealing member which deforms in a manner in which it constricts its diameter adjacent its fore end, apply inwardly directed radial force on the anchors and spears.

In accordance with still another embodiment of the invention, the engaging portion of the sealing member is truncated such that when it is engaged with the blood vessel, the sealing member bears at an inclination over the blood vessel. Accordingly, the fasteners are axially graded, giving rise to an imaginary path extending between edges thereof, said path conforming with the truncated edge of the sealing member.

The sealing device of the present invention is deployable into its operative position by a deploying assembly comprising a tubular housing, a tubular controller received within the housing and a tubular pusher member received within the controller, coaxially received with one another; said pusher adapted for manipulating the sealing member into its activated position.

By manipulating the controller and pusher of the deploying assembly, the sealing member is displaced into the puncture site. The pushing member is fitted with activating surfaces which are fitted with gliding surfaces inclined so as to engage with the manipulating bits at their constricted position and where rotation of the pusher member entails sliding displacement of the manipulating bits about said activating surfaces thereby deploying the anchors into their operative post.

According to one particular embodiment the pusher member also activates the tissue grabbing means into engagement with tissue of the blood vessel surrounding the puncture.

However, in accordance with the embodiment in which the fasteners are spontaneously laterally displaceable, the pushing member of the deploying assembly does not interfere with deployment of the fasteners into engagement with the internal surface of the blood vessel.

The present invention calls also for a method for sealing a puncture in a blood vessel, the method comprising the following steps:

(a) Obtaining a sealing assembly comprising a sealing member fitted within an associated deploying assembly, with a sleeve extending trough the sealing assembly, said sleeve defining a through-going path;

(b) Introducing a medical guide tube through the path;

(c) Removing the sleeve;

(d) Carrying out a medical procedure through the guide tube;

(e) Slidingly displacing the sealing assembly over the guide tube until a fore end of the deploying assembly engages the blood vessel;

(f) Expelling the sealing assembly from the deploying assembly and introducing anchor fastener members of the sealing assembly into tie blood vessel through the puncture;

(g) Deploying anchor fastener member into an operative position in which they are laterally expanded and engage the internal surface of the blood vessel;

(h) Disengaging the sealing assembly from the deploying assembly and withdrawing the guide tube, allowing a resilient sealing member formed in the sealing member to spontaneously seal.

According to a particular embodiment, at step (d) the guide tube is inserted into the blood vessel at an angle corresponding with a truncation angle of an engaging potion formed at the sealing member, for bearing against an external surface of the blood vessel.

Furthermore, at least step (e) is radio-monitored, whereby at least a fore end of the housing is made of or fitted with a radio-opaque material.

According to a modification of the invention, after the device has been deployed into the puncture site, tissue grabbing means are engaged with the tissue surrounding the puncture and upon applying thereto an inwardly directed radial force, the puncture size is decreased. The tissue grabbing means are either internal spikes e.g. formed on the fasteners and adapted for engaging an internal wall surface of the blood vessel, or spear-like member for engaging external wall surface of the blood vessel, or a combination of both means.

According to another particular embodiment, where the fasteners are fitted with rearwardly facing spikes, after step (e) axial force is applied to the sealing member in a rearward direction, thereby having the spikes engage tissue of the blood vessel.

In accordance with a different embodiment of the present invention, there is provided a sealing device for sealing a puncture in a blood vessel, the device is adapted for sliding over a guide tube and comprises a cylindrical body member having a distal end and a proximal end; an axially resilient member extending at said distal end, for bearing against external surface of the blood vessel adjacent boundaries of the puncture; and an anchoring assembly at said proximal end for engaging internal wall surface of the blood vessel; said anchoring assembly comprises at least one flexible anchoring member having a constricted position for insertion into the puncture and a laterally expanded, anchoring position; and a sealing member spontaneously sealable upon withdrawal of the guide tube.

The cylindrical body may be formed with a complete enveloping wall (i.e. a tubular member) or, in accordance with a modification of the invention, may be a cylindrical cage formed with an apertured wall or a framework, or a plurality of support struts.

The axially resilient member ensures that the anchoring assembly projects only a required minimum into the blood vessel so as to minimize blood flow disturbances.

Said at least one flexible anchoring member is spontaneously deployable into its anchoring position for engagement with an inside surface of the blood vessel, adjacent boundaries of the puncture. The arrangement is such that during introducing the sealing device into the puncture location, the at least one anchoring members are in their constricted position and upon penetration through the puncture into the blood vessel, the at least one anchoring member deform into the anchoring position, bearing against the inside surface of the blood vessel.

The anchoring assembly comprises at least one anchoring member anchored to the body adjacent the proximal end of the housing with a free end thereof facing the distal end of the housing, said anchoring members are resilient members formed so as to normally project laterally into their anchoring. The anchoring members may be integrally formed with the housing or fixedly attached thereto. According to one application the anchoring members are in the form of a plurality of flexible leaf-like members which may be formed in a variety of different forms e.g. pointed or rounded edges, etc. According to another application the anchoring assembly may be in the form of a resilient skirt-like member, possibly formed with one or more axial slots for increasing flexibility of the one or more anchoring member. The sealing member, according to one particular design, is a membrane made of resilient material formed with a central aperture which is normally biased into a sealed position. The aperture may be punctured or a recessed, allowing introduction of a guide tube through the aperture. Typically but not necessary, the sealing member extends at or adjacent the proximal end of the housing of the sealing device.

According to a different design, the sealing member is a resilient sleeve-like member received in the housing with a circumferential portion sealingly secured at a proximal end, and flattened adjoining lips at the distal end, whereby fluid flow through the sleeve, in a direction from the distal end towards the proximal end, is not admitted. Even more so, fluid pressure on outside walls of the sleeve cause the lips to tighten against one another. According to an embodiment thereof, side edges of the lips are secured to the housing adjacent the distal end. The sealing member is designed to admit introduction of the guide tube there through.

The axially resilient member is formed, by one embodiment with an oblique fore end. It is thus advantageous that where more than one anchoring member is provided that they extend at a corresponding inclination as that of the fore end of the axially resilient member, i.e. such that a circumferential gap between said fore end and the anchoring members is uniform. One possible way to render the resilient member axial deformity is by designing it as a bellows-type device.

By still a further application of the invention there is provided a deploying assembly for introducing the sealing device into the puncture vicinity and for deploying it into the operative, sealing position. The assembly comprises a rigid elongate retractor having a tube-embracing portion slidable over the guide tube and extending between a fore end and a rear end with an expanding member fitted at the rear end for radially expanding or constricting the tube-embracing portion. The retractor device is used for penetrating through tissue layers and retraction thereof. The deploying assembly further comprises a pusher sleeve slidable over the guide tube and within the retractor, with a fore end thereof engageable with the proximal end of the sealing device so as to propel it through the rector and into the puncture of the blood vessel. The fore end of the pusher sleeve and of the retractor may be radio opaque for monitoring its progress of the deploying procedure.

According to an embodiment of the device, the tube-embracing portion is formed with one or more axial openings to allow introducing or removal of the guide tube there through. According to one particular design, the tube-embracing portion is formed of two or more shell-like portions readily displaceable.

In some instances it is desirable to provide an obturator for inserting through the guide tube during the sealing process. The obturator serves to rigidify the guide tube and prevent it from accidental collapse, and where the obturator is provided with a plurality of radio-opaque markings, it also facilitates monitoring the deployment process of the sealing device.

A method utilizing the above different embodiment and the deploying member comprises the steps of:

(a) Obtaining a sealing assembly comprising a sealing member and an associated deploying assembly;

(b) Introducing a medical guide tube through a path within the sealing device;

(c) Carrying out a medical procedure through the guide tube;

(d) ounting a retractor of the deploying member over an exposed portion of the guide tube;

(e) slidingly displacing the retractor over the guide tube into through tissue layers until a fore end of the retractor engages the blood vessel;

(f) expanding the retractor and slidingly displacing the sealing device over the guide tube and through retractor, into the vicinity of the puncture of the blood vessel;

(i) deploying the sealing device into its operative state within the puncture wherein the resilient member is depressed against external surface of the blood vessel and the anchoring members spontaneously deploy to their operative position in which they are laterally expanded and engage the internal surface of the blood vessel;

(j) withdrawing the deploying member and the guide tube, allowing a resilient sealing member of the sealing member to spontaneously seal.

Where the deploying assembly comprises a pusher member, displacement of the sealing device into the vicinity of the puncture (step (f)), will be carried out by said pusher member.

### BRIEF DESCRIPTION OF THE DRAWINGS

For better understanding the invention and to see how it may be carried out in practice, reference will now be made to the accompanying drawings, illustrating in a non-limiting manner, some embodiments of the present invention, in which:

FIG. 1 is an isometric view of a deploying assembly fitted with a sealing member (not seen) in accordance with the present invention, the deploying assembly being at its initial state;

FIG. 2 is a sectional view of the device seen in FIG. 1, visualizing also the sealing member;

FIG. 3A is a partially sectioned side elevation of the sealing device in accordance with the present invention, in a constricted, non-operative position;

FIG. 3B is a top view of FIG. 3A, illustrating a rear end of the sealing device;

FIG. 3C is a bottom isometric view of the device seen in FIG. 3A;

FIG. 4A is a top isometric view of tile sealing member, with the anchors removed;

FIG. 4B is an isometric view of an anchor of the sealing device of the in accordance with an embodiment of the present invention;

FIGS. 5A, 5B and 5C correspond with FIGS. 3A, 3B and 3C, respectively, in the operative state of the sealing device;

FIG. 6 is an isometric view of the housing of the deploying assembly in accordance with the present invention;

FIG. 7 is an isometric view of a controller of the deploying assembly in accordance with the present invention;

FIG. 8A is an isometric view of a pusher of the deploying assembly in accordance with the present invention;

FIG. 8B is a front elevation of the fore end of the pusher member seen in FIG. 8A;

FIG. 9 is an isometric view of a sleeve of the deploying device in accordance with the present invention;

FIGS. 10A-10E represent four consecutive steps of deploying a sealing device in accordance with the present invention, using a deploying assembly of the invention;

FIG. 11A is a top isometric view of a sealing device in accordance with another embodiment of the invention, the device in its non-operative state;

FIG. 11B is a bottom isometric view of the device seen in FIG. 11A;

FIG. 11C is a planar view of a fore (bottom) end of the device of FIG. 11A;

FIG. 11D is a planar view of a rear end of the device of FIG. 11A;

FIG. 11E is an isometric view of a spear received within the sealing device of FIGS. 11A-11D;

FIG. 12 is an isometric view of a modification of a pusher member used with a sealing device according to the second embodiment illustrated in FIGS. 11A-11E;

FIG. 13A is a side elevation of the sealing device seen in FIGS. 11A-11D, in the operative state of the device;

FIGS. 13B-13D correspond with FIGS. 11B-11D, respectively, in the operative state of the device;

FIG. 14 is an exploded isometric view of a sealing device in accordance with a different embodiment of the invention;

FIG. 15A is an isometric view from below illustrating the sealing device of FIG. 14 with the anchors in the constricted position;

FIG. 15B is an isometric view from below illustrating the device of FIG. 14 in a first deployment position in which the anchoring means are deployed into the blood vessel for engagement with the internal surface of the blood vessel;

FIG. 15C is an isometric view from below illustrating the sealing device of FIG. 14 in an operative position in which the anchors dispositioned radially inwardly;

FIGS. 16A-16C are side views corresponding with FIGS. 15A-15C, respectively;

FIG. 17 is a isometric view illustrating the sealing device of FIG. 14, with the anchors in their constricted position, the tubular sealing envelope removed;

FIG. 18 is an isometric view from above of a sealing device in accordance with a different embodiment of the present invention;

FIG. 19 is a partially sectioned isometric view of the sealing device seen in FIG. 18;

FIG. 20 is a partially sectioned isometric view illustrating the sealing device of FIGS. 18 and 19 mounted on a guide tube;

FIG. 21 is a partially sectioned isometric view of an embodiment of the sealing device seen in FIGS. 18 and 19;

FIG. 22 is an isometric view from below visualizing the sealing member of the embodiment of FIG. 21;

FIG. 23 is a partially sectioned isometric view illustrating a sealing device similar to the previous embodiment with the sealing member being superimposed of the embodiment illustrated in FIGS. 19 and 21;

FIG. 24 is a partially sectioned isometric view illustrating the sealing device of FIG. 22 mounted on a guide tube;

FIG. 25 is still another embodiment of a sealing device in accordance with the invention, wherein:

FIG. 25a is a top isometric view of the sealing device in it's sealing position;

FIG. 25B is bottom isometric view of the sealing device in it's sealing position (or free position); and

FIG. 25C is a top isometric view of the sealing device mounted on a guide tube in a position in which the device is deployed into the vicinity of a puncture;

FIG. 26A is bottom isometric view of a sealing device according to still a different embodiment, anchoring members thereof being at their reacted position;

FIG. 26B is the same as FIG. 26A, the anchoring members illustrated in their expanded position;

FIG. 27 is an exploded isometric view of a deploying assembly for facilitating deployment of a sealing device in accordance with the present invention; and

FIGS. 28A-28F are consecutive steps illustrating the deployment of a sealing device in accordance with the present invention with the use of the deploying device of FIG. 27 after carrying out a surgical procedure.

### DETAILED DESCRIPTION OF SPECIFIC EMBODIMENTS

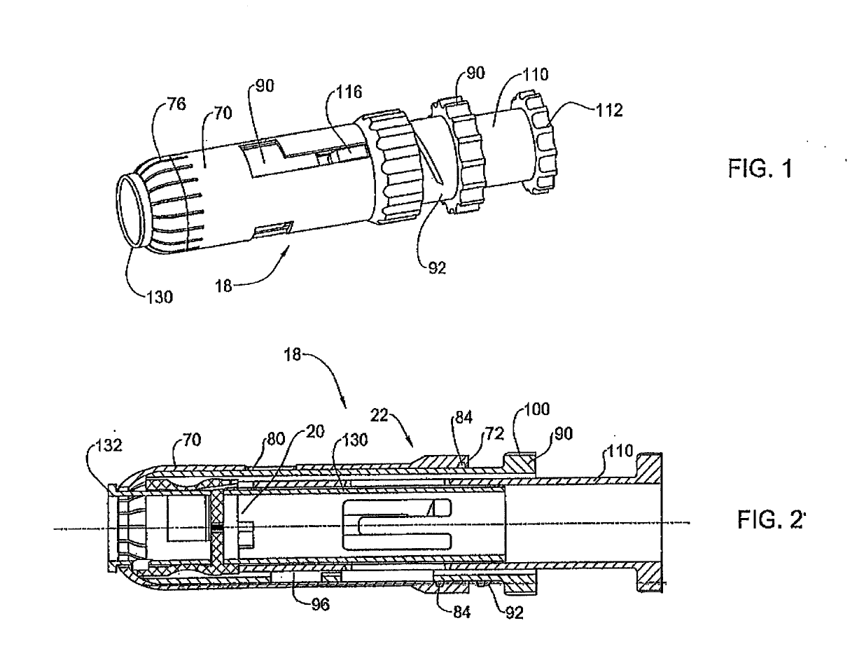

Turning first to FIGS. 1 and 2, there is illustrated an assemblage 18 of a sealing device generally designated 20 (FIG. 2) and a deploying assembly generally designated 22.

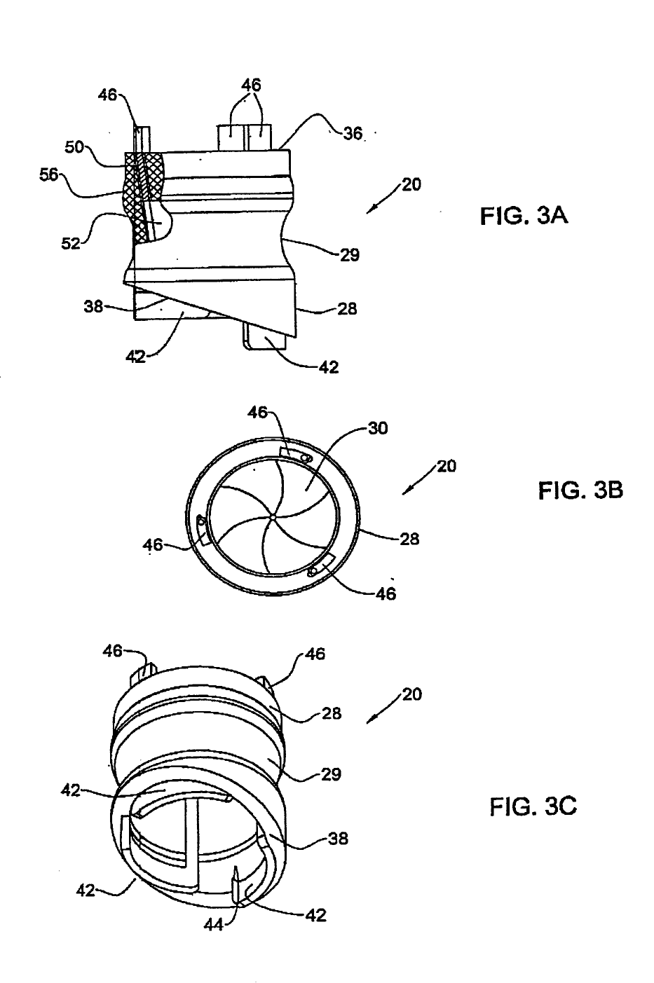

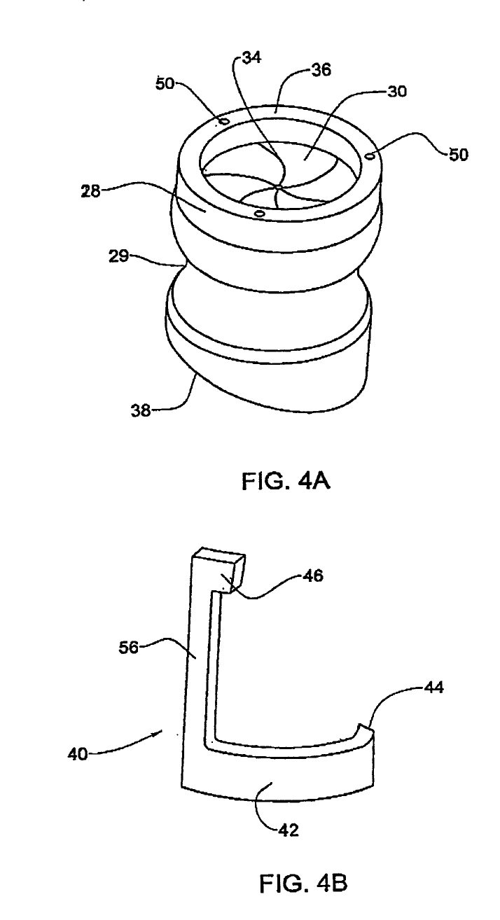

Further reference will be made to FIGS. 3 and 4 for exemplifying the sealing device 20. The sealing assembly 20 consists of a sealing member 28 having a generally tubular section formed of a resilient material, e.g. silicon rubber. The sealing member 28 is fitted with a sealing portion 30 which is a diaphragm formed with a plurality of vein-like slots 34. These slots may have different shapes, e.g. plain radial slots, etc. The diaphragm 30 is normally biased to acquire an essentially sealed state. However, any other type of sealing arrangement is possible instead of diaphragm 30.

An intermediate portion 29 of the sealing member 28 is significantly resilient and is axially collapsible upon applying thereto some axial force.

The sealing device 28 has a rear end 36 and a fore end 38 which is a truncated engaging portion adapted for bearing against the external surface of the blood vessel (not shown) and this arrangement is intended for conforming with an insertion angle of typically between about 30° to 45° which the angle at which a medical procedure of inserting an angioplast stent or other procedure is taking place, as known per se.

It is to be appreciated that the sealing member 20 may be formed as a unitary item or may be assembled out of several components. For example, the sealing member may be molded out of several different components, each having different resiliency, etc. Alternatively, the sealing member may be a formed by means of a resilient member received within a housing imparting it regions of varying resiliency as may be required.

The sealing member 20 is fitted with three anchors 40 (FIG. 4B) each formed with a fastener 42 for engaging with an internal surface of the blood vessel. Each fastener 42 is formed at its end with a spike 44, the purpose of which will become apparent hereinafter. At a rear end of the anchor 40 there is formed a manipulating bit 46.

Sealing member 28 is formed, in the particular example, with three bores 50 extending at an essentially radially direction (FIG. 3A) from the rear end 36 of the sealing member 28 at a slight inclination inwards. The bores 50 extend only a limited potion of the sealing member 28 and then opens into a common cavity marked 52 of the sealing member 28. A stem portion 56 of anchors 40 is received within bore 50 with the manipulating bits 46 extending above rear end 36 of sealing member 28 and fasteners 42 extending in a graded manner, giving rise to an imaginary path which is essentially parallel to the engaging edge portion 38. This arrangement ensures that when the sealing member 20 is in its activated position engaged within a puncture of a blood vessel, it extends at an oblique angle with essentially similar pressure applied by the three fasteners 42.

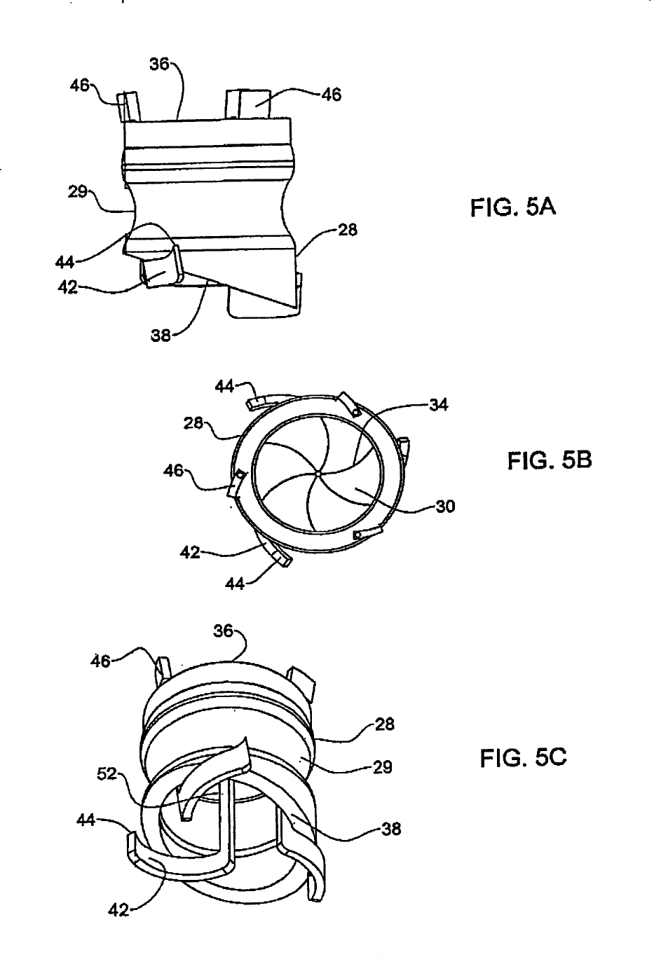

The arrangement is such that a stem portion of each of the anchors 40 is rotatably received within bores 50 between an inoperative position in which the fasteners 42 are constricted and do not extend from the tubular structure of sealing member 28, and an operative position in which the fasteners are laterally expanded for engagement with internal surfaces of the blood vessel, in the position shown in FIGS. 5A to 5C. However, it is to be noted that the stem portion 56 of anchors 40 extend through a common space 52 within the sealing member 20.

The manner in which the fasteners are shifted into their laterally expanded position will be explained hereinafter with reference to the deploying assembly.

As seen in FIG. 2, the sealing device 20 is received within a fore end of the deploying assembly 2.

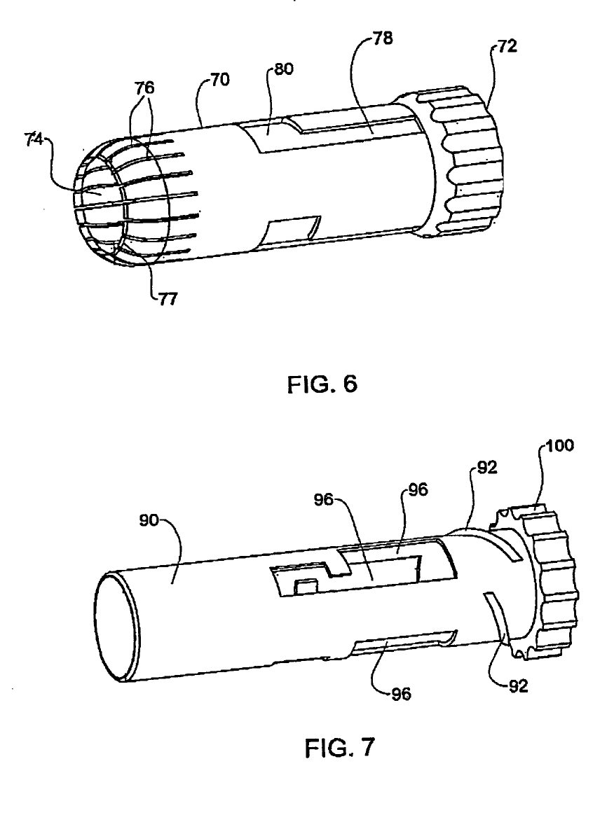

The deploying assembly comprises a housing 70 (FIG. 6) which is a tubular member formed with an open rear end 72 and an open front end 74. A front portion of housing 70 narrows towards the front end of the housing and is axially slotted at 76, rendering said front portion resilient whereupon the sealing device 20 may be expelled therethrough as will become apparent hereinafter. Housing 70 is further formed with a plurality of axial slots 78 (three in the present case) extending axially with a lateral expansion 80 at a fore end of the slot. As can further be seen in FIG. 2, a rear end potion of housing 70 is formed with an internal thread 84 which is adapted for thread engaging with a corresponding thread formed on a connector member 90 (FIG. 7) rotatably engaged within housing 70, whereby rotation of connector 90 yields axial displacement within housing 70.

According to a preferred embodiment of the invention, at least a fore end 77 of the housing 70 is made of or fitted with a radio-opaque portion.

Turning now also to FIG. 7, the controller 90 is illustrated, said connector having a tubular shape snugly receivable within housing 70 and formed with a thread 92 (see also FIGS. 1 and 2) engageable within tread grooves 84. In the particular example the threading is a three-start thread formed with a high pitch. The controller 90 is formed with three openings 96 corresponding with slots 78 and extensions 80 thereof Controller 90 is fitted at its rear end with a grip potion 100.

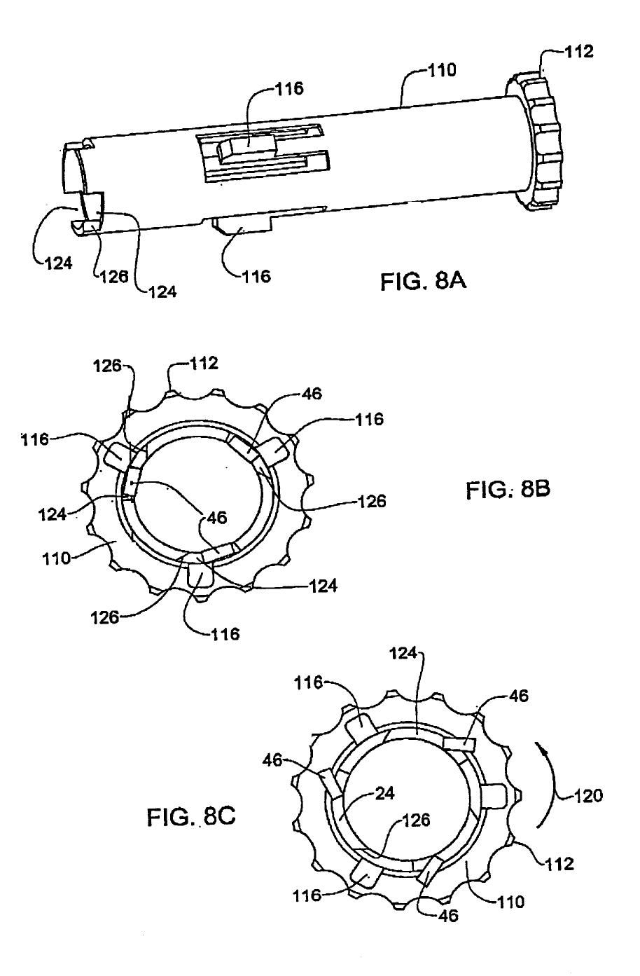

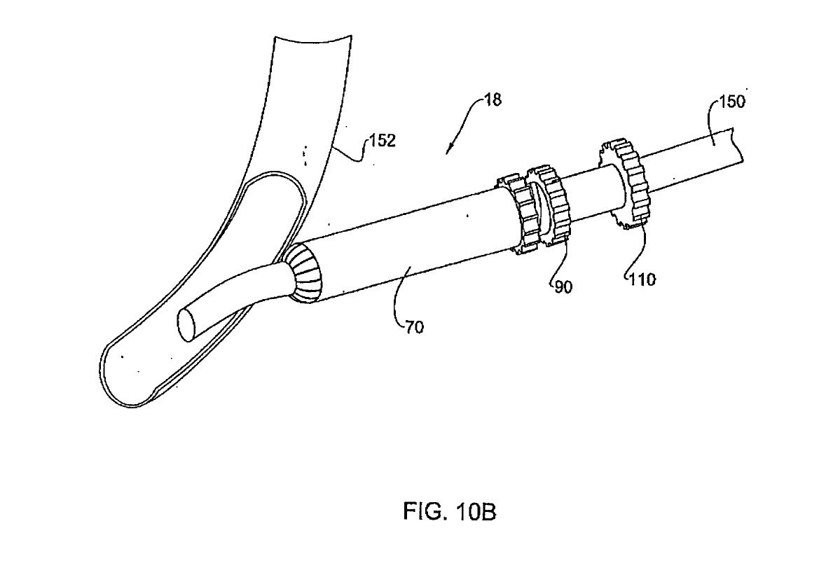

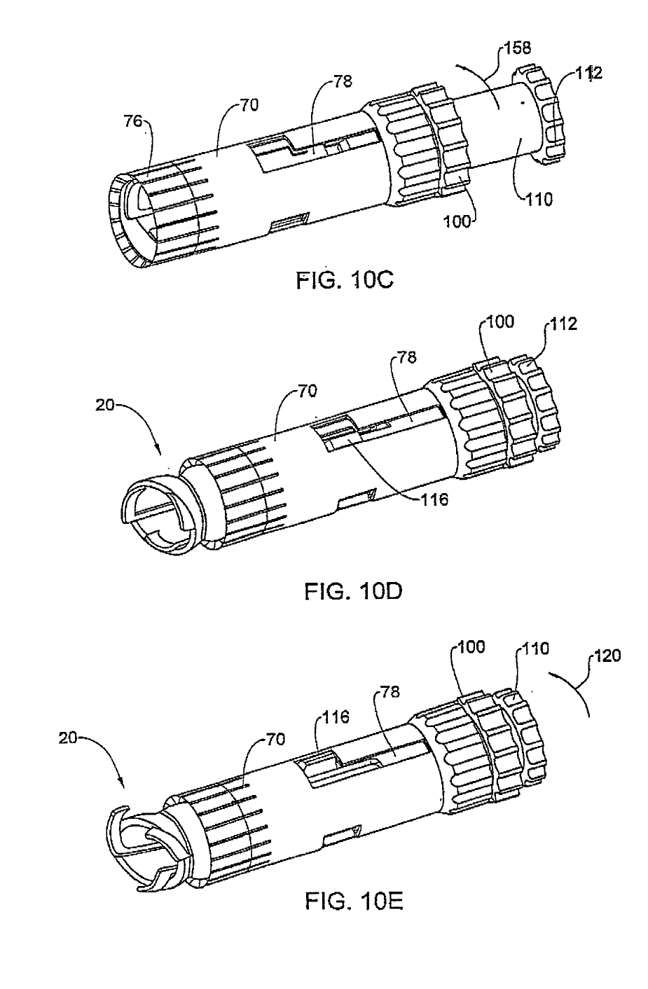

A pusher member 110 (FIG. 8A) is received within controller 90 and is adapted for axial displacement therewithin. Pusher 110 is formed with a ribbed gripping end 112 and three lateral bulges 116 equi-angularly disposed and adapted for projecting through apertures 96 in controller 90 and recesses 78 in housing 70. The arrangement is such that bulges 116 ensures that the pusher 110 is restricted only to axial displacement with respect to the housing 70 which axial displacement is restricted to the length of recess 78. However, when bulges 116 reach portion 80 of recess 78, the pusher member may be rotated to a limited extent, as represented by arrow 120 in FIG. 10D.

Turning now also to FIG. 5B, a fore end of the pusher 110 is illustrated in which three recesses 124 are formed giving rise to a wall 126 cut at a slant serving as a gliding surface. Recesses 124 are sized to accommodate the manipulating tip 46 of anchors 40 whilst surfaces 126 are engageable with said manipulating bits.

At an initial state of the assemblage 18 (FIG. 8B) the recesses 124 of pusher 110 accommodate the manipulating bits 46 in a manner exemplified by one bit only in FIG. 8B illustrated in a solid line. However, upon rotation of the pusher 110 in the direction of arrow 120 (FIG. 8C) thereby imparting axial rotation of anchors 40 within the sealing member 28.

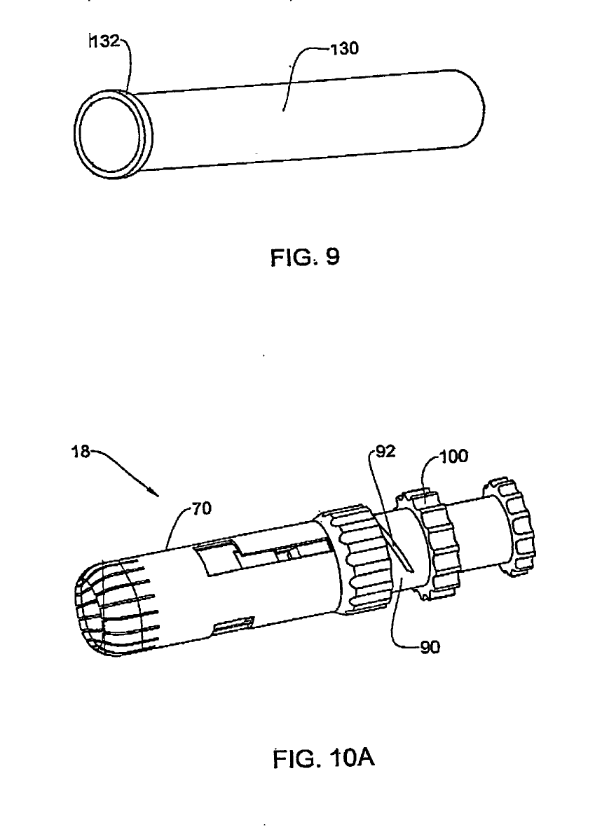

Turning now back to FIGS. 1 and 2, and with further reference to FIG. 9, a tubular sleeve 130 is inserted into the assemblage 18 through a fore end thereof, via opening 74 of the housing. The inner diameter of sleeve 130 is adapted to accommodate a guide tube (also referred to as “sheath”) used for carrying out the medical procedure, e.g. during angioplasty.

A fore end of sleeve 130 is fitted with an annular rim 132 for restricting its insertion into the housing and to constitute a gripping member.

In order to avoid mishandling of the device, directing arrows (not shown) are provided on the various components of the deploying assembly, indicating the direction of correct handling. At this stage pusher 110 remains fixed and does not displace with respect to housing 70.

Further attention is now directed also to FIGS. 10A-10E. The assemblage 18 seen in FIG. 2 is mounted on a guide tube (sheath) 150 seen in FIG. 10B and then the sleeve 130 is removed by pulling it at the rim 132. Removing the sleeve 130 causes the sealing device 20 to slidingly engage the guide tube 150. In FIG. 10A, the assemblage 18 is illustrated after having removed the sleeve 130. At this stage, the assemblage 18 is slidingly received over the sheath 150 and the medical procedure, e.g. an angioplasty, takes place by introducing the guide tube 150 into blood vein 152 (FIG. 10B). Typically, the guide tube 150 is introduced into the blood vein at an angle of between about 30 and 45°, depending on the particular medical procedure.

At a next step, after completing the medical procedure, whilst the guide tube 150 is still within blood vessel 152, the physician pushes the sealing assemblage 18 through tissue layers (skin, muscle, etc.) towards the blood vessel 152 by gripping the housing 70 and sliding it along the guide tube 152. This procedure takes place under radiography whereby the radio-opaque portion 77 is visible so that the physician may determine when the fore end of the housing 70 reaches the blood vessel 152.

Then, whilst gripping the housing the controller 90 is rotated by gripping it at grip 100 in a direction of arrow 158 in FIG. 10C until fully received within housing 70, thus entailing its axial displacement forwardly within the housing 70. This results in expelling the sealing device forward within the housing 70 giving rise to expanding of the fore end 76 of the housing 70, as seen in FIG. 10C.

After expanding of the fore end of housing 70 took place, pusher 110 is axially displaced forwardly (FIG. 10D) whereby bulges 116 move within grooves 78 to their forward most position. This maneuver exposes the sealing device 20 as can be seen in FIG. 10D. Upon forward expelling of sealing device 20 the oblique engaging portion 38 of sealing member 28 comes to bear against the external surface of blood vessel 152 whereby further displacement of the device into the blood vessel entails shrinkage of the resilient sealing member 23, in particular at the resilient portion 29, resulting in displacement of the lower portion of the shanks 56 and fasteners 42 of anchors 40 with the blood vessel.

In order to anchor the sealing assembly within the puncture of the blood vessel, the pusher 110 is rotated in the direction of arrow 120 in FIG. 10E, allowing bulges 116 to radially displace within the broadened opening 80.

By this rotation, the anchors 40 are axially rotated into the position seen in FIGS. 5A-5C whereby the fasteners are laterally expanded for engaging internal wall surface of the blood vessel at peripheral portions of the puncture formed in the blood vessel.

The physician then applies light force in an outward direction ensuring that spikes 44 engage within the tissue of the blood vessel whereby the device is properly anchored and secured within the puncture of the blood vessel. At a final stage the guide tube 150 is withdrawn allowing the diaphragm 30 to spontaneously close, thereby sealing the sealing device preventing blood flow through. It then remains only to further remove the deploying assembly 22, thereby completing the medical procedure and the puncture in the blood vessel is sealed.

The arrangement is such that the shrinkage of the sealing member 28, in particular at the intermediate portion 29 entails inward radial deflection of the anchors 40, thereby applying some inward radial force urging a constriction of the size of the puncture in the blood vessel for more rapid spontaneous healing thereof.

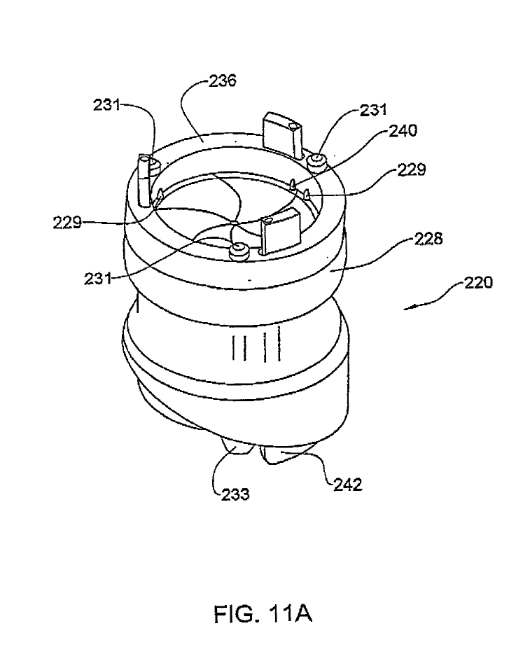

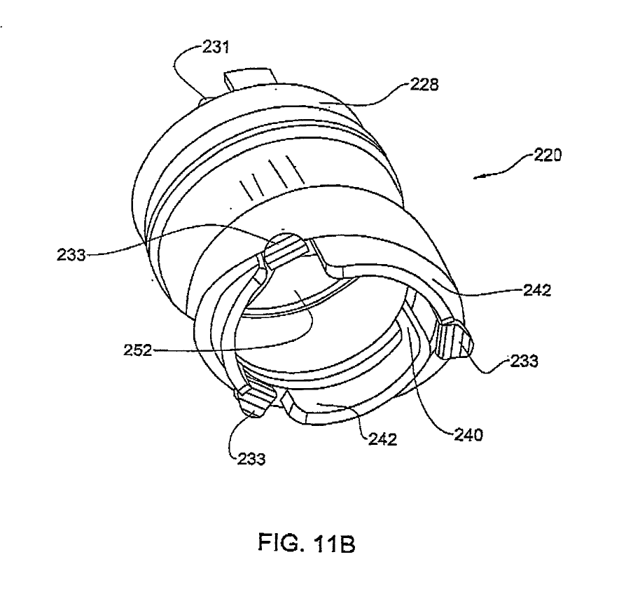

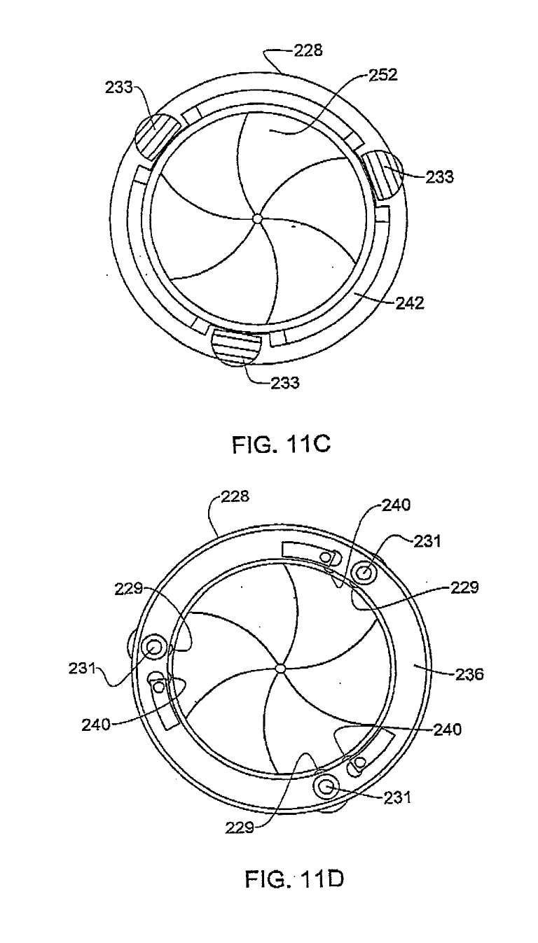

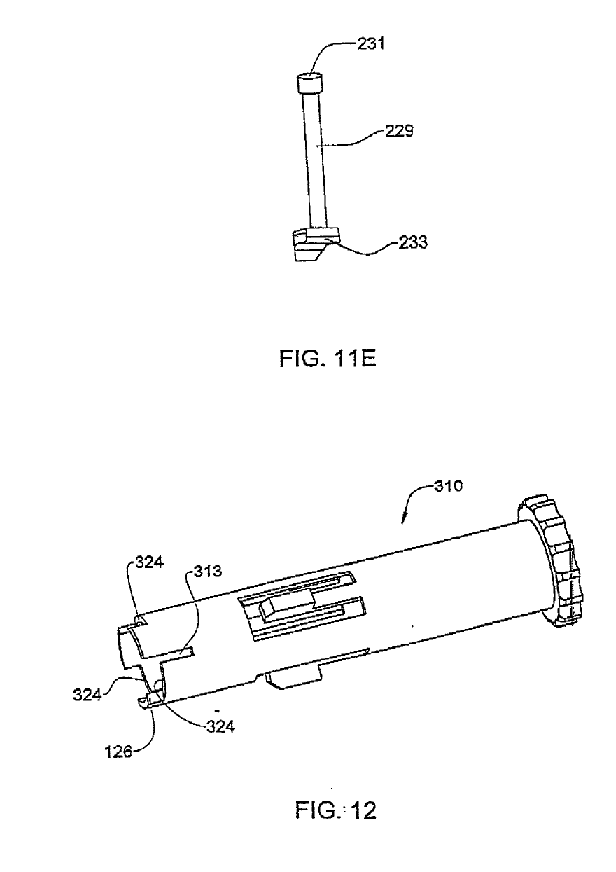

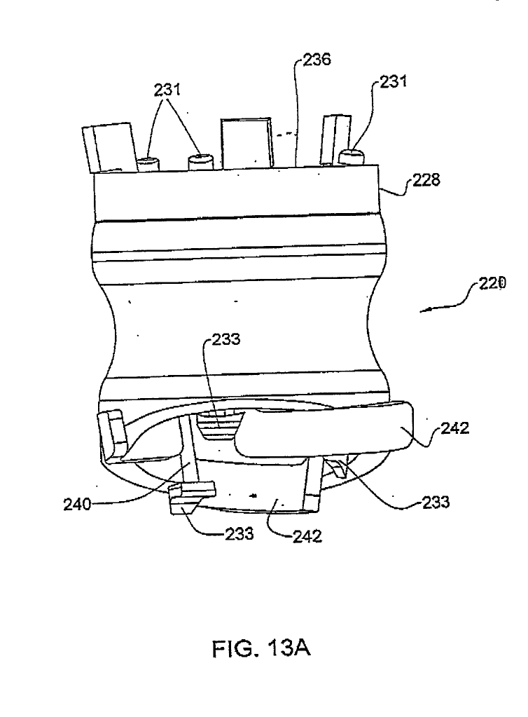

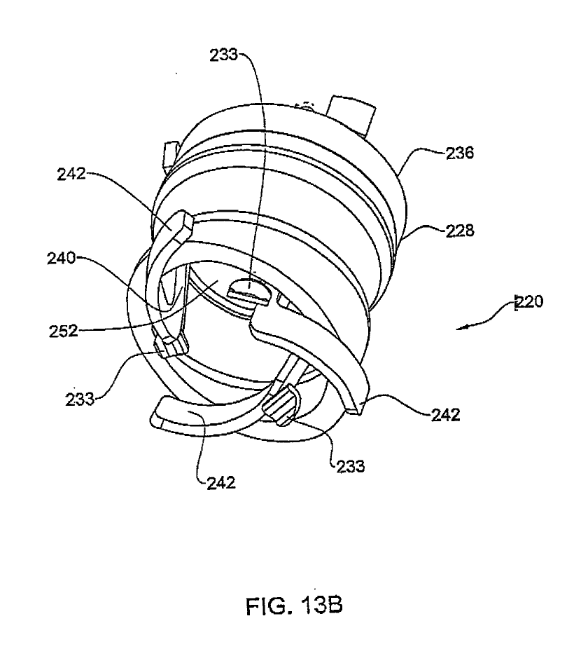

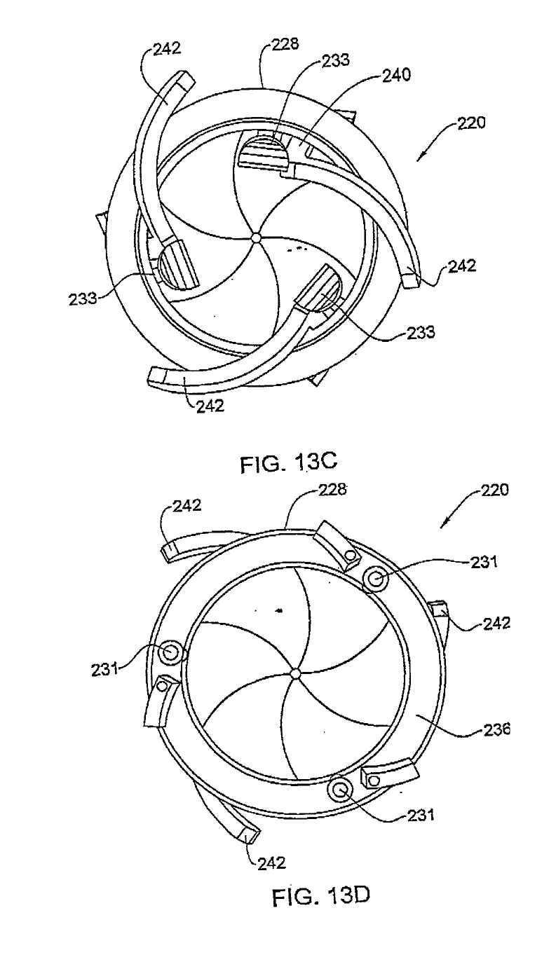

Further attention will now be made to another embodiment of the invention, making reference to FIGS. 11 through 13, illustrating a modified sealing assembly 220 which in FIGS. 11A through 11D is in the non-operative position and in FIGS. 13A through 13D is at an operative position.

The sealing assembly 220 comprises a sealing member 228 which is essentially similar to sealing member 28 in the embodiment of the previous drawings, with the only exception being that it comprises a plurality of bores (three in the present example), thus, elements which are similar to elements disclosed in connection with the previous embodiment will be designated with corresponding reference numbers shifted by two hundred.

The bores are not visualized in the figures and they axially extend only through a rear portion of sealing member 228. The bores extend from a rear end 36 into a common central cavity 252 (FIGS. 11B, 11C and 13A-13C) of the sealing member 228.

Received within the bores are three spears 229 (FIG. 11E), each having a rear head portion 231 of greater diameter than the bores of the sealing member, thus preventing the spears 229 from unintentional disengagement from the bores. A fore end 233 of each of the spears 229 is pointed such that the edges of the spears define together an imaginary circular path. A rear stem portion of the spears 229 axially extends Through the bore of the sealing member and a fore stem portion of the spear extends in the common space and is radially displaceable inwardly, upon deformation of the sealing member 228.

The sealing device 20 is also fitted with three anchors 240, similar to anchors 40 in the previous embodiment with the only difference residing in that the fasteners 242 lack a rearwardly facing spike 44 and are rather essentially flat though being arranged in a grading pattern, as in the previous embodiment.

The associated pusher member 310 (FIG. 12) for interacting with the sealing device 220 of the present embodiment, is principally similar to pusher 110 of the previous embodiment with an exception that it is formed with three axial recesses 313 each extending from a recess 324. The longitudinal recess accommodates the rear heads 231 of the spears 229 when the pusher member 310 is in its forward position, thus preventing the spears 229 from engaging with tissue.

Operation of the sealing member disclosed hereinabove in connection with the second embodiment is principally similar to that which has been explained and illustrated in connection with the previous embodiment, but with the following exception. After introducing the sealing device 220 into the vicinity of the puncture in the blood vessel and manipulating the deploying member as disclosed with reference to the first embodiment the fasteners 242 radially expand as in FIGS. 13A-13D (similar to the arrangement of corresponding FIGS. 5A-5C) apart from the spikes. Instead, fore end 232 of spears 229 grabbingly engages into external wall surface of the blood vessel and upon axial deformation of the resilient sealing member 228, the fore ends of the spears are forced in a radially-inward direction, thus adjoining the lips of the puncture at the blood vessel and restricting the puncture's size.

It will be appreciated that the fasteners may also be pointed with spikes, as in the first embodiment, for the same purpose.

Herein the description, reference was made to three spikes and three spears. It is to be appreciated that this is an example only and any other suitable number of tissue-grabbing means may be used.

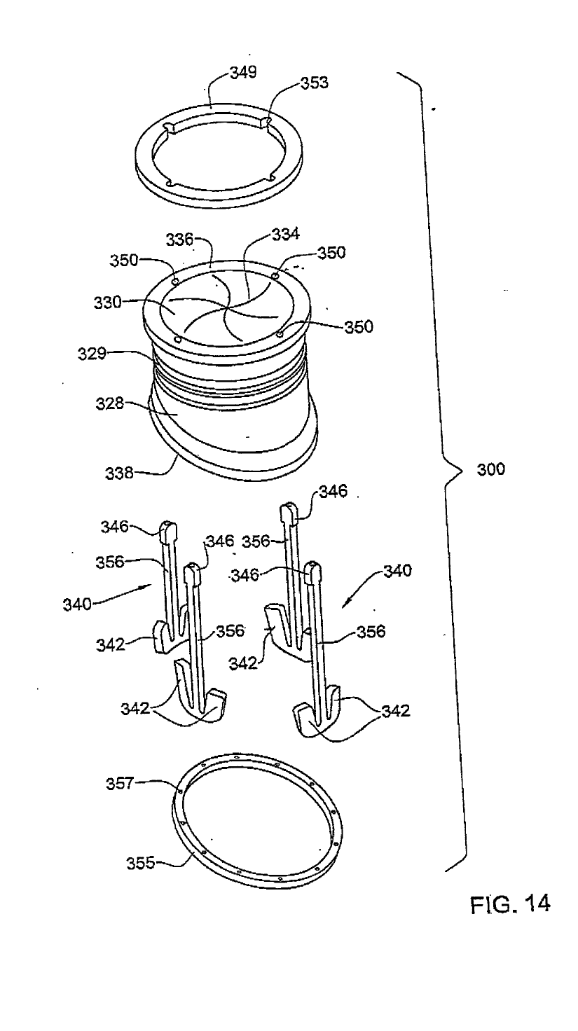

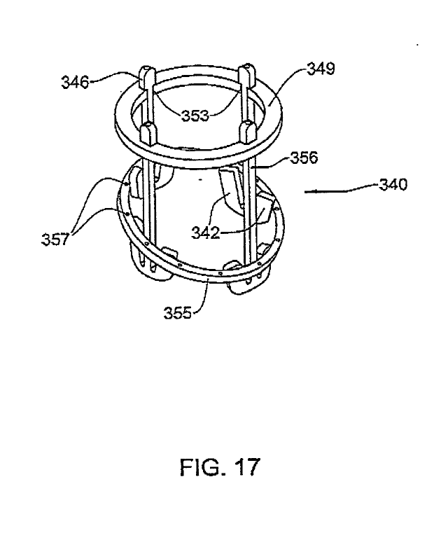

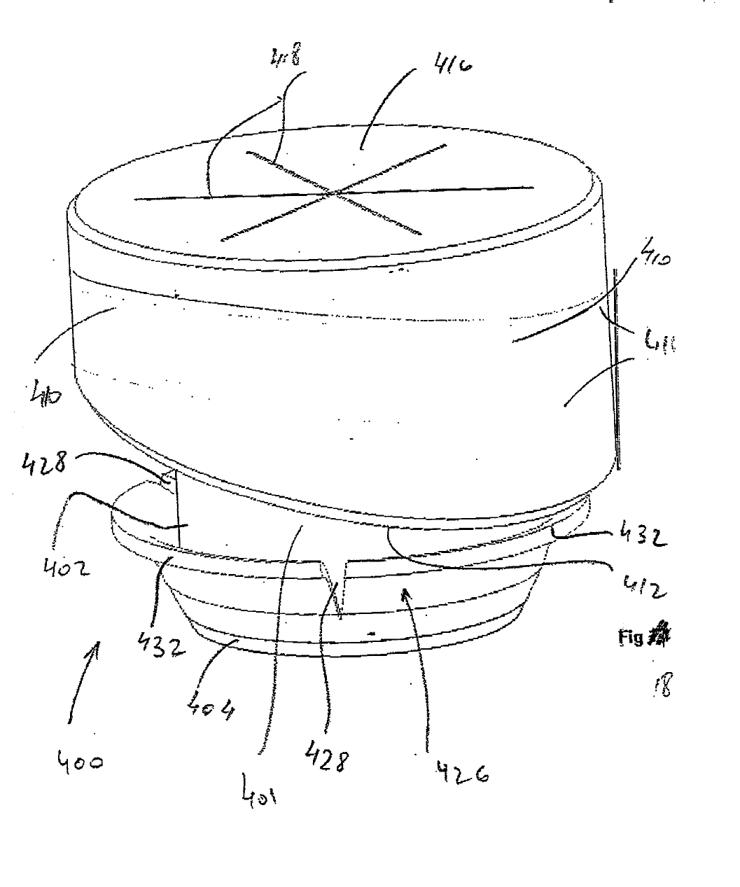

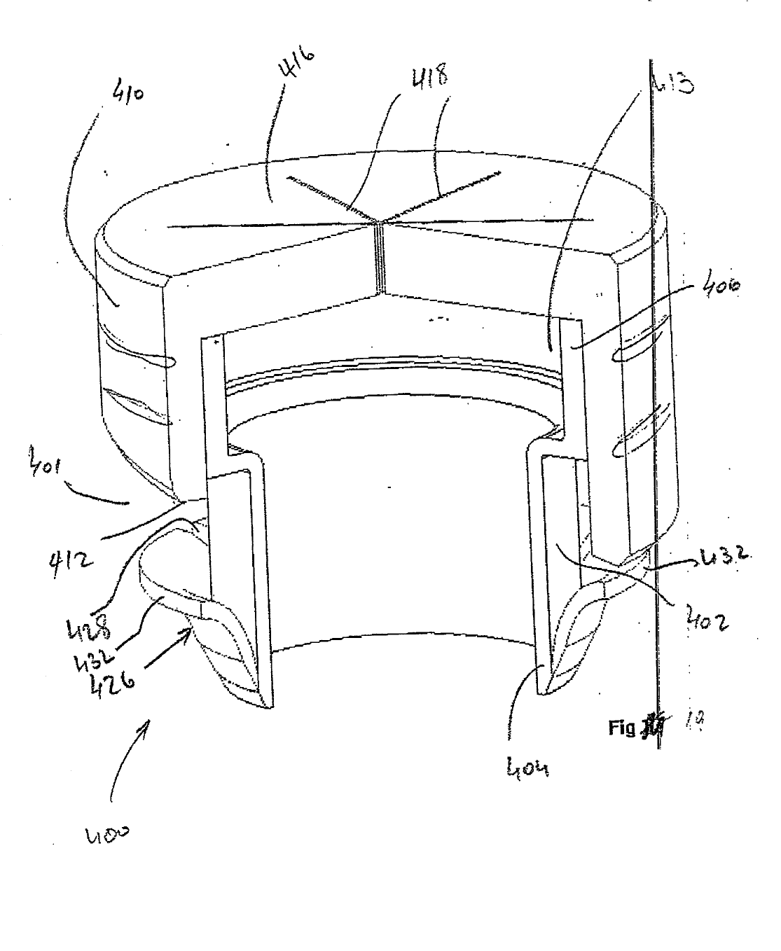

Further attention is now directed to FIGS. 14 to 17 illustrating a sealing device in accordance with another embodiment of the invention generally designated 300. For the sake of clarity, those components which are similar to components of sealing device 20 illustrated in connection with the embodiments seen in FIGS. 3 and 4 are given same reference numerals shifted by 300.

The sealing device 300 comprises a sealing member 328 formed at a rear end thereof with a sealing portion 330 in the form of a diaphragm fitted with a plurality of vein-like slots 334 which as normally biased into a sealed position though allowing introducing therethrough of a sheath, as explained hereinbefore.

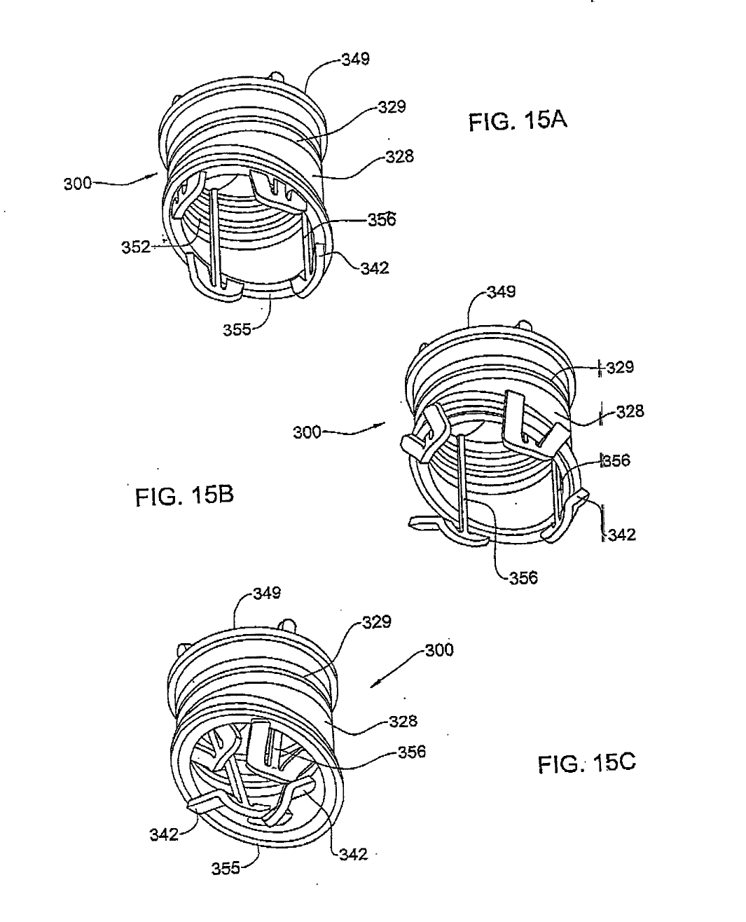

The sealing member 328 has a tubular shape with an intermediate resilient portion 329 with four bores 350 axially extending from rear end 336, through a rear portion of the side walls and into the central, common space 352 (FIGS. 15A-15C). The structure of sealing member 328 is made of a resilient material such as silicone rubber which may be easily deformed and which assumes its original shape as soon as depressing force is removed.

Attached onto rear edge 336 there is a retaining ring 349 made of an essentially rigid material and formed with four recesses 353 corresponding with apertures 350 on rear edge 336 of the sealing member 328.

A fore end 338 is truncated as explained in connection with a sealing device 28 in the previous embodiment, and is fitted with a anchor retention ring 355 made of an essentially rigid material, the purpose of which will become apparent hereinafter. Retention ring 355 is formed with a plurality of bores 357 for improving the connection with the sealing member 328 during the molding process. Retention ring 355 may be made of a radio or pack material.

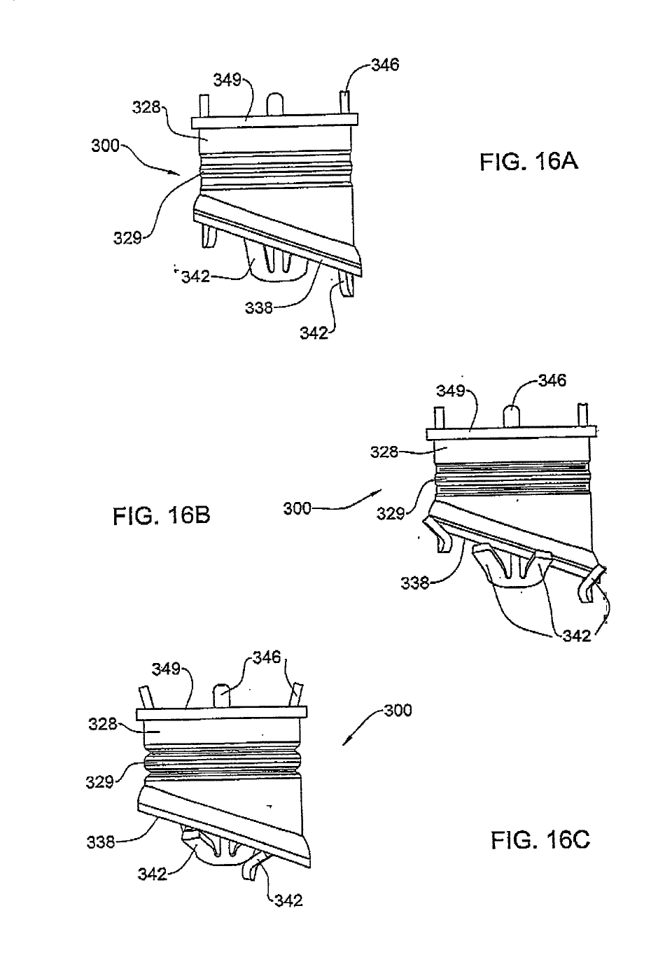

Four anchors 340 are provided, each fitted at a rear end thereof with an expanded head 346, larger than apertures 353 in ring 349. Each anchor 340 is formed at its fore end with two fastener 342 which in the present embodiment are of different lengths. The fasteners 342 are illustrated in FIG. 14 in their contained position in which they extend essentially parallel to stem portion 36 and assume an arced shape corresponding with the radii of the sealing member 328 so as to blend with it in the constricted position as will be explained hereinafter with reference to FIG. 15A. However, the fasteners 342 are normally biased to displace in a lateral direction as illustrated in FIGS. 15B, 15C and corresponding FIGS. 16B and 16C. This arrangement is obtained for example by pre-straining the fore end of the anchors.

Preferably all components are made of a biodegradable or bio-absorbable material.

Turing now to FIGS. 15A and 16A, the sealing member 300 is illustrated in the constricted position in which the stems 356 of anchors 340 coaxially extend along the sealing member 328 and when a guide sheath (not shown) extends through the sealing member 300 it depresses the stem portions 356 and the fasteners 342 into the resilient sealing member 328, as illustrated in FIG. 15A, whereby the sealing device 300 is slidable over the sheath. This is the position in which the sealing member is retained within the deploying assembly (similar to that disclosed in connection with the previous embodiment, whereby a medical procedure is carried out without interference of the sealing device 300.

Assembly of the sealing device over the guide tube (sheath) is similar with that disclosed in connection with the previous embodiment. After completing the medical procedure, whilst the guide tube is still within the blood vessel, the physician pushes the sealing assemblage through tissue layers (skin, muscle, etc.) towards the blood vessel, sliding it along the guide tube. This procedure takes place under a geography whereby the radio or pack portion (typically retention ring 355) is visible, so that the physician may determine when the fore end of the sealing device 300 reaches the external face of the blood vessel.

Then, similar to the description in connection with FIG. 10C above, whilst gripping the housing of the deploying member, control 90 is rotated, resulting in expelling of sealing device 300 in a forward direction, giving rise to expansion of the fore end 76 of housing 70 (FIG. 10C).

After expansion of the fore end, pusher 110 is axially displaced forwardly (FIG. 10D) exposing the sealing device 300 until the oblique fore end 338 bears against the external surface of the blood vessel such that further axial displacement of the sealing device 300 entailed axial shrinkage of the resilient sealing member 328 (as in the position of FIGS. 15B and 16B), in particular along the resilient portion 329, until a position in which anchors 342 disengage from the retention ring 355 and laterally project with the blood vessel, engaging with inner surface of the blood vessel, at peripheral portions of the puncture formed in the blood vessel.

Preferably all components are made of a biodegradable or bio-absorbable material

As in the previous embodiment, the physician then applies a light force in an axial outward direction, first to ensure that the fasteners 342 are indeed engaged with the tissue of the blood vessel, whereby the device is properly anchored and secured within the puncture of the blood vessel. Then, the guide tube is withdrawn, allowing the sealing diaphragm 320 to spontaneously close, thereby sealing the sealing device and preventing blood flow therethrough.

Upon removal of the guide tube and the deploying assembly, an inward radial force is applied by the walls of the sealing member 328, applying an inward radial force over the stem portion 356 of anchors 340, entailing their radially inward displacement as in the position of FIGS. 15C and 16C urging constriction of the size of the puncture in the blood vessel for faster healing and spontaneous sealing of the puncture.

It will be appreciated that the particular construction of the fore end of pusher 110 (namely recesses 124 and gliding surfaces 126) are unnecessary, nor is the arrangement of rotation of the pusher 110 within the controller 90. In the present embodiment there is merely required axial displacement of the components.

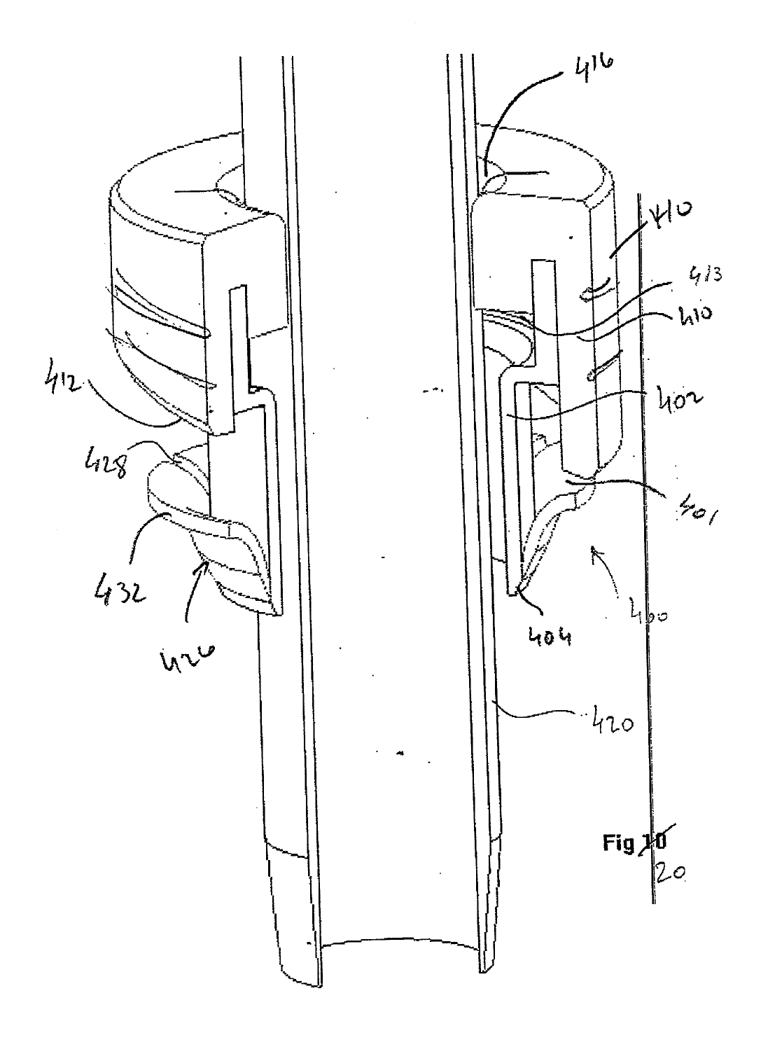

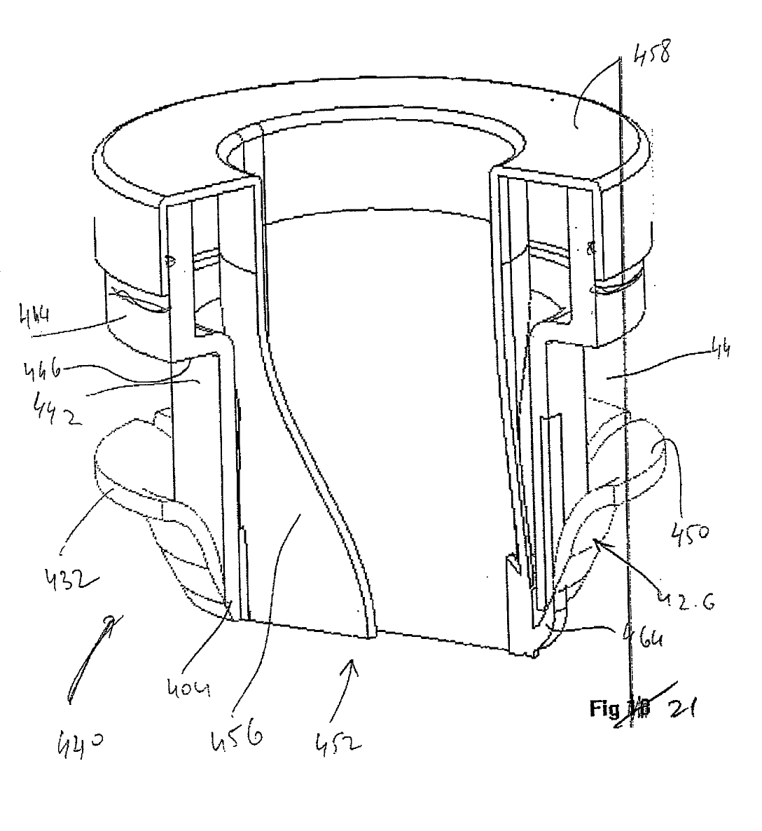

Turing now to a further embodiment illustrated in FIGS. 18 and 19, there is a sealing device generally designated 400 comprising a tubular body 402 having a distal end 404 and a proximal end 406, with a axially deformable head member 410 axially resilient and for that purpose it is typically made of silicon rubber and may be formed as a bellows, so as to be axially deformable at integral fold lines 411. Optionally, the head member may be provided with an axially biasing spring member embedded within the device so as to increase resiliency of the device (not shown). The head member has an oblique distal end 412 which as explained in connection with previous embodiments, is suited for inclined engagement with a blood vessel. A proximal end of the member 410 serves as a sealing member 416 formed with a plurality of radially extending slits 418. This arrangement makes it possible to introduce through the sealing device a guide tube 420 (FIG. 20) whilst upon removal of the guide tube 420 the sealing member 416 spontaneously seals as in the position of FIGS. 18 and 19.

It is to be appreciated that rather than radial slits 418 there may be provided merely a through-going aperture and owing to significant resilience of the sealing member 416 it is possible to introduce a guide tube there through while maintaining the feature of spontaneous sealing thereof.

The sealing device 400 is formed at its distal end with an anchoring assembly 426 which in the present embodiment is a ring-like member formed with several axial slits 428 giving rise to formation of flexible anchoring members 432 which in the non-biased position have a free end thereof laterally projecting as in FIGS. 18 to 19. The anchoring member blends with the end 404 of the housing 402 which slightly taper so as to facilitate penetration of the sealing device into the puncture at the blood vessel.

Arrangement is such that the distal end 412 of member 410 is adapted for bearing against external surface of the blood vessel adjacent boundaries of the puncture whilst the resilient anchor members 432 bear against inside boundary surfaces of the punctured vessel whereby resilience of the member 410 and of the anchoring-members 432 ensure tight sealing of the apertures boundaries which are maintained clamped between the peripheral surface of distal end 412 and the anchoring members 432.

Whilst in the disclosed embodiments the housing 402 is a complete enveloping wall (i.e. a tubular member) it is appreciated that it may rather be in the form of a cylindrical caged formed out of a cylindrical framework or may be an apertured tubular member. Furthermore, the housing may be somewhat tapering such that a fore end thereof is narrower then its rear end. The gap 401 formed between member 410 and anchoring member 426 is in fact a clamping zone sized so as to clampingly accommodate a wall thickness of the punctured vessel. It is thus desired that some axial biasing means be provided and this is obtained by either or both axial flexibility of member 410 and the elasticity of anchoring member 432. The contour of anchoring members 432 is such as to facilitate easy insertion through the apertures perimeter and spontaneous expansion of the anchoring members to the anchoring position, presenting withdrawal of the device.

The housing 402 has at its rear (distal) end a widened portion indicated in FIG. 19 as 413 for receiving flaps of the sealing membrane 416 at the open position when a guide tube extends there through, as in FIG. 19.

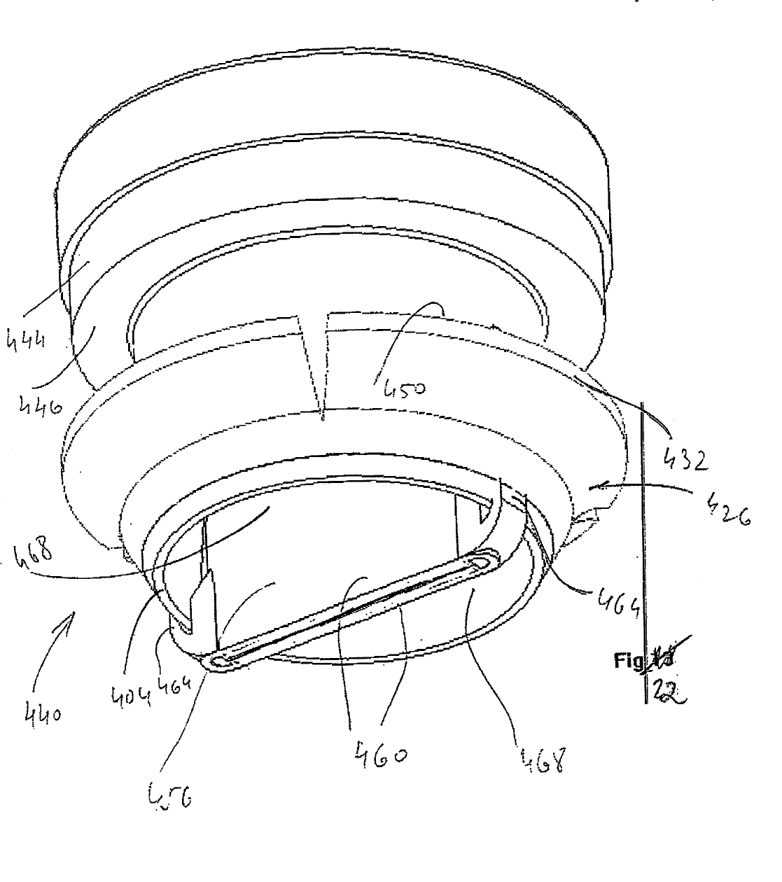

The embodiment of FIGS. 21 and 22 illustrates a sealing device generally designated 440 which is principally similar to the previous embodiment except that the sealing arrangement is different and is devoid of the axially deformable head member. Instead, housing 442 comprises a laterally projecting portion 444 with an annular bottom surface 446 bearing against the external surface of the vessel adjacent the boundaries of the puncture consisting a gap 448 between said surface 446 and the three edges 450 of anchoring members 432.

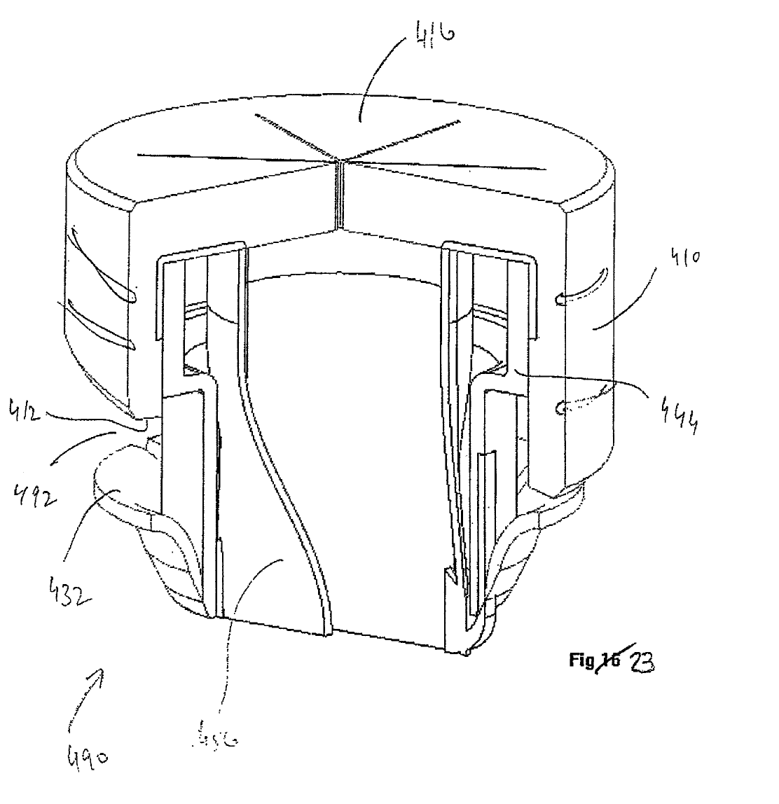

The sealing mechanism in accordance with the present embodiment, generally designated 452 comprises a sleeve-like member 456 which at the proximal end comprises a skirt portion 458 attached to the housing 442 and sealingly secured thereto and at the distal end comprises two flattened lip members 460, spanning across the diameter of the housing. The sleeve 456 is made of a flexible material which permits introduction therethrough of a guide tube 480 (see FIG. 24) and the sleeve spontaneously gains the position of FIGS. 21 and 22 in which the lip 460 sealingly engage one another, preventing flow through the sealing device 440.

As further noted, best in FIG. 22, the sleeve member 456 is secured at the distal end 404 of the housing by means of attachments 464 extending below the anchoring member 426 thus preventing collapsing of the sleeve member 456 upon withdrawal of the guide tube 480 (FIG. 24). Furthermore, two hollow pockets 468 are formed at both sides of the sleeve 456, whereby upon withdrawal of the guide tube 480, when the sealing device is in its operative position within a puncture in a blood vessel, blood enters these pockets and applies pressure on the lips 460, tightening their engagement thus increasing sealing of the sealing device.

With further reference to FIG. 23, there is illustrated a sealing device 490 which is a combination of sealing devices 400 (FIGS. 18-20) and 440 (FIGS. 21 and 22). Accordingly, like elements have been given like reference numbers as in the previous embodiments. The sealing device 490 comprises a head member 410 which is axially resilient and comprises a sealing portion 416. A sealing gap 492 is formed between an oblique bottom surface of head member 410 and an upper free end of anchoring members 432. In addition to sealing member 416 there is provided a sealing sleeve 456 clampingly secured between a portion of housing 444 and the head member 410, the operation of said sleeve member being as explained hereinabove in connection with the embodiment of FIGS. 21 and 22.

FIG. 24 illustrates he sealing device 490 of FIG. 23 with a guide tube 480 extending therethrough. At this position the medical procedure is carried out through the lumen of guide tube 480 whilst upon withdrawal of the guide tube 480, the sealing device spontaneously seals, first upon engagement of lips 460 of sleeve 456 and then, sealing of the sealing member 416 upon removal of the guide tube 480 from head member 410.

Whilst in the disclosed embodiments the housing 402 is a complete enveloping wall (i.e. a tubular member) it is appreciated that it may rather be in the form of a cylindrical caged formed out of a cylindrical framework or may be an apertured tubular member.

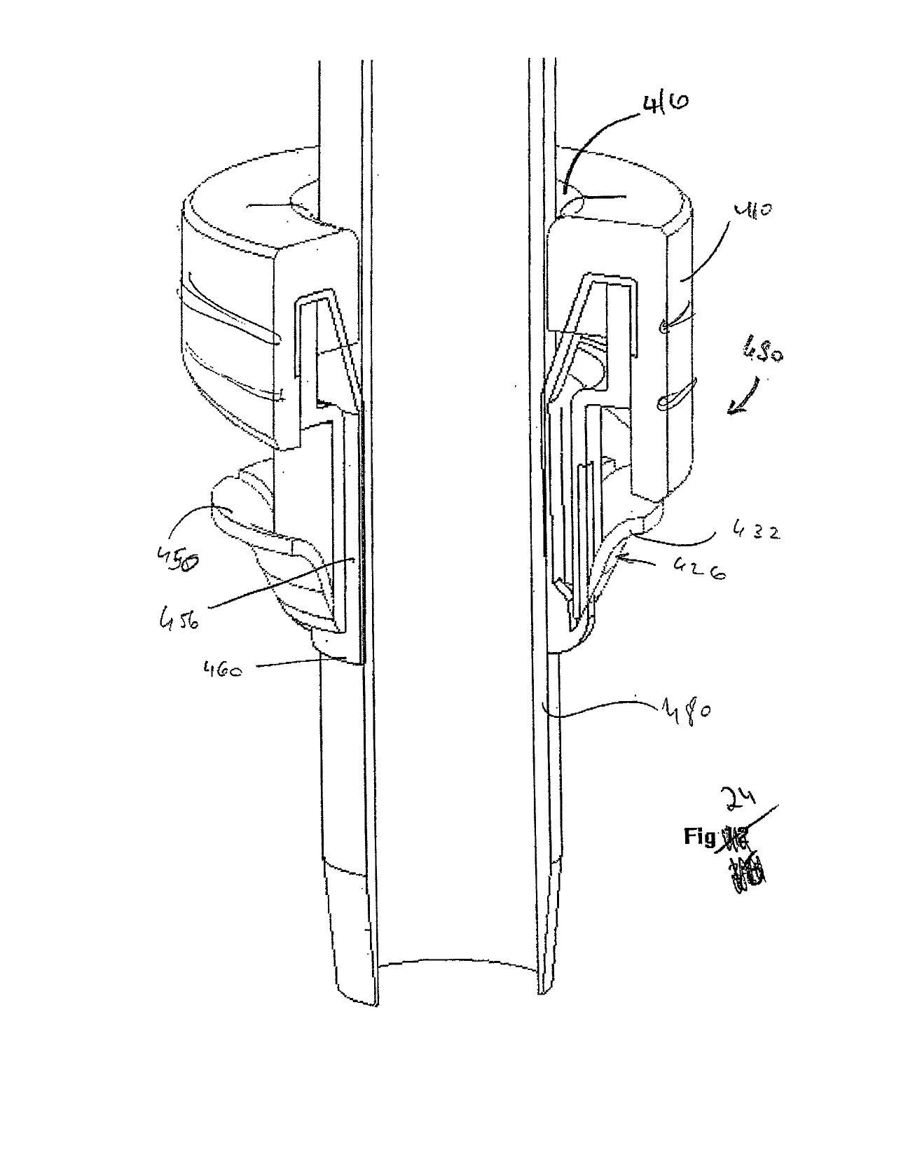

The sealing device 493 of FIG. 25 differs from the sealing device 400 in FIG. 18 only in its anchoring assembly 494 constructed by forming a plurality of apertures 495 (FIGS. 25A and 25B) in the tubular housing 496 and imparting the cut-outs elasticity thereby forming anchoring members 497 which are normally outward projecting as in the figure. As in the previous embodiment, the formed, distal end 498 is slanted so as to facilitate easy insertion of the sealing device into the puncture's vicinity. In operation, the anchoring members 497 will bear against an inner wall surface of the blood vessel at peripheries of the puncture formed therein. In the figures the tips of the cut-outs are rounded, however they may also be pointed, etc (not shown). The sealing device 494 in FIG. 25C is illustrated mounted on a guide tube 499.

The sealing assembly in the embodiment of FIGS. 25 is similar as in FIG. 18 and comprises a body member 410 formed with a sealing assembly 416. However, other sealing arrangements are possible as well or in combination, e.g. the arrangement as in FIGS. 21 and 22.

The embodiment of FIG. 25 has an advantage when used in conjunction with a sealing assembly as illustrated in connection with the embodiment of FIGS. 21 and 22, whereas blood may flow through apertures 495 formed in housing 496 thus applying pressure on the sealing sleeve (not shown in FIG. 25) to tighten the sealing effect.

It will be appreciated by a skilled person that the anchoring assembly may, rather than be integrally formed with the housing, may be fixedly attached thereto, e.g. by adhering, welding, etc.

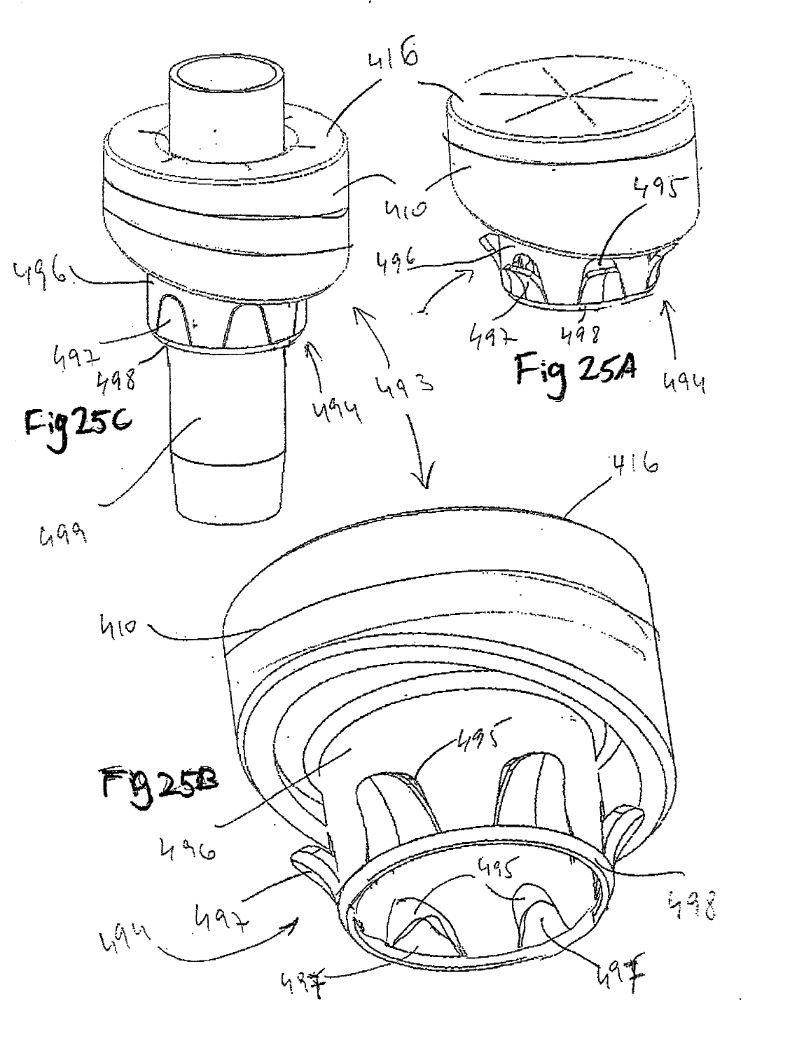

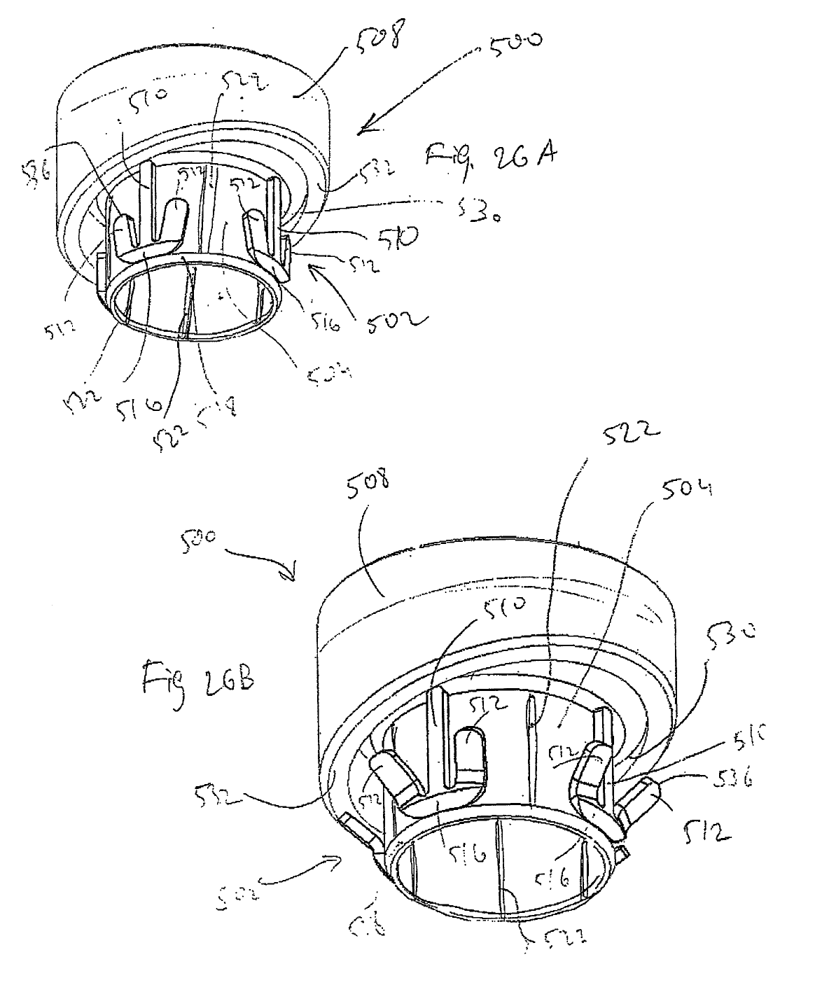

Further attention is now directed to FIGS. 26A and 26B illustrating still another embodiment of a sealing device in accordance with the present invention generally designated 500 which being compared to the previous embodiments differ mainly in the different anchoring assembly generally designated 502 as well as in the construction of the tumor member 504.

In the embodiment of FIG. 26, the head member 508 is identical with corresponding head member 410 in the embodiment of FIG. 25, and is integrally formed with a sealing portion (not shown). The anchoring assembly 502 comprises several anchor units each supported by a study 510 formed at its proximal end with two anchors 512 which are resilient members formed so as to normally laterally project as in FIG. 26B although may be easily deformed into the position of FIG. 26A, e.g. while being pushed through an aperture in a blood vessel whereby the walls of the blood vessel cause the anchor members 512 to deform into position of FIG. 26A.

For assisting in the penetration through the aperture in the blood vessel, the distal tip of the anchors designed as 516 is tapered and blends with a corresponding tapering end 518 of housing 504.

The housing 504 rather than being a tubular member as in the previous embodiment is a cage-like member constructed of a plurality of support studs 522 bearing at their distal end the ring 518. The density of stud 522 determines the rigidity of the structure.

Further noticed in the embodiment of FIGS. 26A and 26B, the gap 530 between the fore end 532 of head member 508 and the distal end 536 of the anchoring members 512 remains con t in spite of tie inclination of the fore end 532 of head member 508 since the studs 510 of the anchoring assembly are of corresponding length thereby assuring that when the sealing device 500 is introduced into site at an angle (which is typical procedure in carrying out the medical procedures concerned) the sealing of the puncture is most effective.

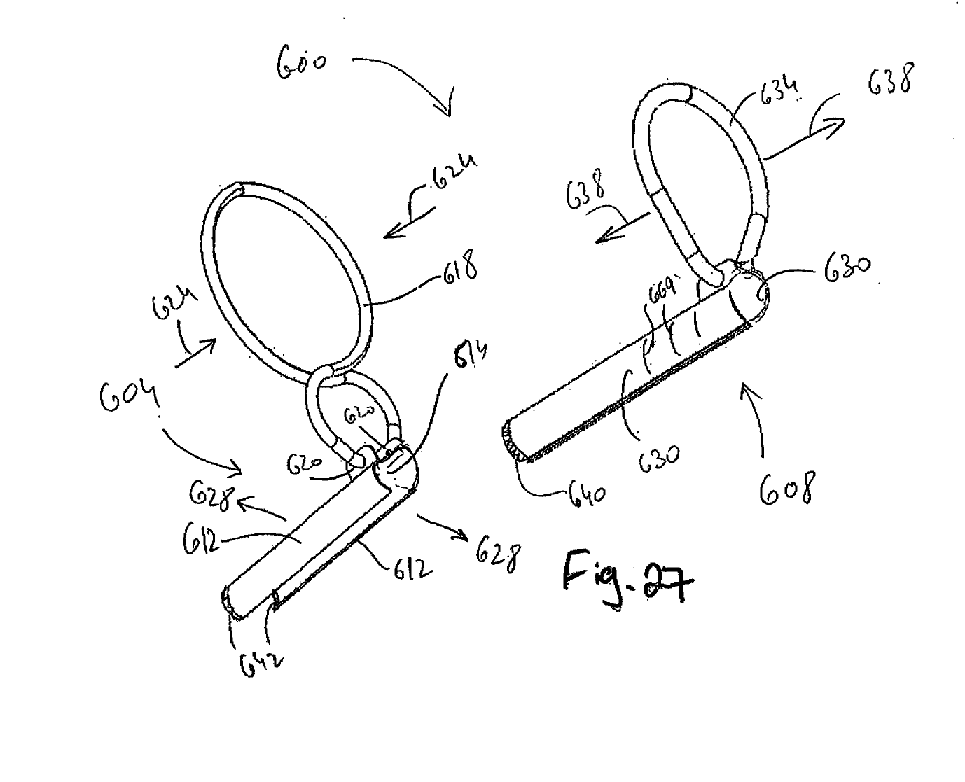

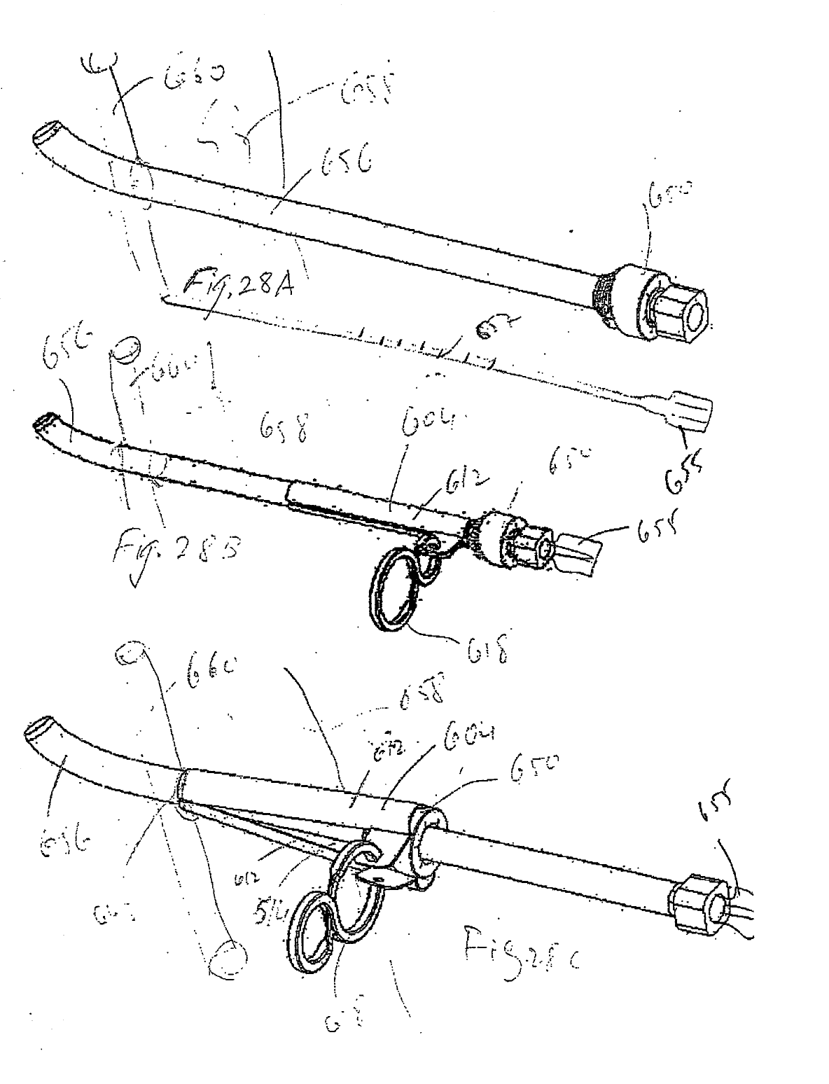

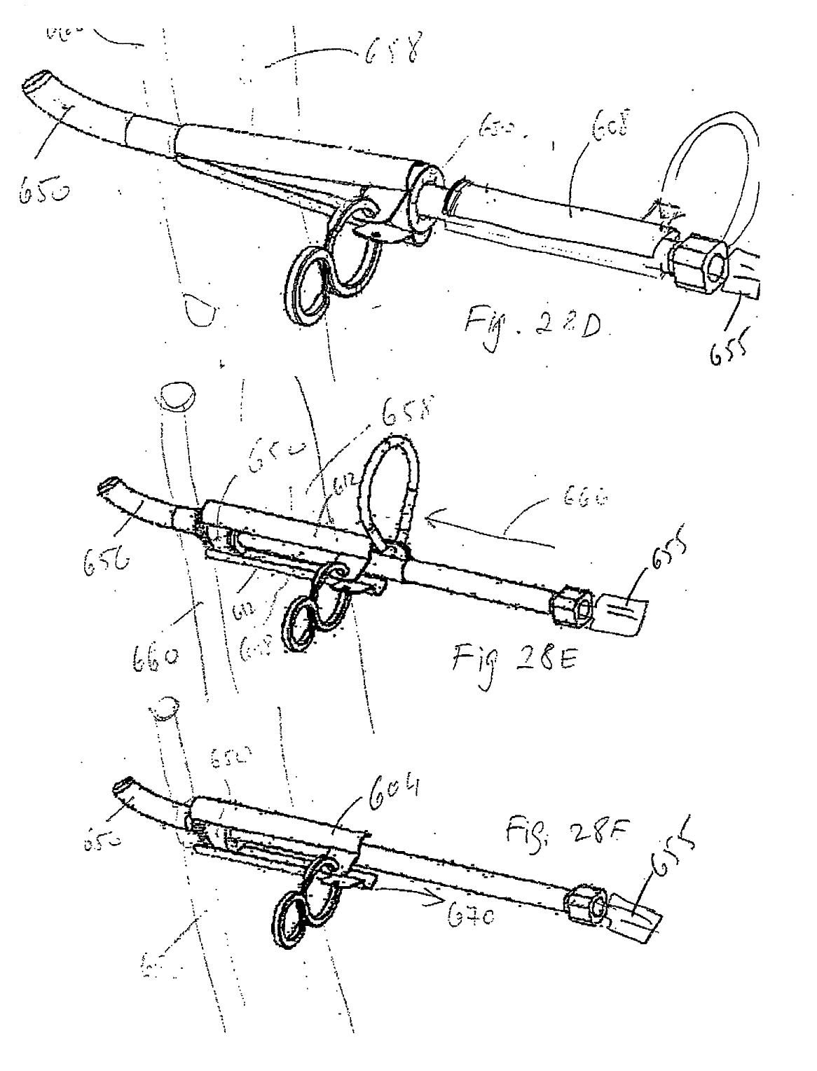

FIG. 27 illustrates a deploying assembly in accordance with another embodiment of the present invention generally designated 600 comprising a retractor unit 604 and a pusher unit 608. Retractor unit 604 comprises two shell members 612, facing each other giving rise to a confined space therebetween 614 sized for slidingly receiving a guide tube as will become apparent hereinafter with reference to FIGS. 28. The shell members 612 are articulated to one another by a coiled spring actuator 618 fixed to the shell members at flanges 620.

The arrangement is such that depressing the actuating member 618 I the direction of arrows 624 entails retraction of shell members 612 in the direction of arrows 628.

The pusher unit 608 is similar in construction to the retractor unit 604 as it comprises two shell-like members 630 sized for sliding over a guide tube (as will become apparent hereinafter with reference to FIGS. 28) and within the cavity 614 of the retractor unit 604. Shell members 630 are retractable from one another by opening the biasing support ring 634 in the direction of arrows 638 so as to enable mounting of the pusher unit 608 on a guide tube.

At least the fore end 640 of pusher unit 608 and possibly also the fore end 642 of retractor unit 604 are radio opaque so as to enable monitoring the deploying procedure by known radiology means.

With further reference being made now to FIGS. 28A to 28F, a method for deploying a sealing device in accordance with the present invention, using a to deploying assembly 600 as illustrated in connection with FIG. 27, will now be described, using the same reference numerals as in FIG. 27, whereby the sealing device is designated 650 which device may be any of the aforementioned devices as described and as claimed in accordance with the present invention.

In FIG. 28A, there is illustrated a guide tube 656 which prior to beginning with a medical procedure is fitted at its rear end with the sealing device 650, the latter having no interference whatsoever with the medical procedure taking place through the guide tube 656 which is introduced through different tissue layers 658 into the blood vessel 660 through a puncture formed therein.

As the medical procedure ends, an obturator 655 is introduced into the guide tube 656 for stiffening same and preventing its accidental collapse. According to one embodiment, the obturator is marked with a plurality of radio-opaque marks 557 for facilitating monitoring the deploying process. Then, the deploying assembly is mounted, first mounting the retractor unit 604. For that purpose, the shell members 612 are separated from one another by depressing the actuator ring 618 expanding the distance between the shell members 612 enabling mounting of same on the guide tube 656, as in FIG. 28B at a location between seating device 650 and the organ's tissue 658.

Reactor unit 604 is then pushed over the guide tube into the tissue layers 658 until its fore end 642 reaches the blood vessel 660, monitoring the progress of the radio opaque fore end 642 under conventional radiology means. Once the retractor unit 604 reached its position, the actuating ring 618 is again depressed (in the direction of arrows 624 in FIG. 27) overcoming the natural sealing force of the tissue layers 658, until a position in which the gap 614 between shell members 612 is sufficient, at least at a rear end thereof to allow introducing of the sealing member 650 as in the position of FIG. 28C.

Once the sealing device 650 is pushed forwards into the position of FIG. 28C (this may be carried out merely by using one's hands) the pusher unit 608 is mounted at the rear portion of the guide tube 656 as in the position of FIG. 28D. By pushing the pusher unit 608 in the direction of arrow 666 in FIG. 28E, the sealing device 650 is propelled forward over the guide tube 656 and between the shell members 612 of the retractor 604. As the proximal leading end of the staling device 650 penetrates through the puncture of the blood vessel 660 its anchoring assembly will deform into a retracted position and will spontaneously snap into its anchoring position as explained hereinabove in connection with the sealing devices. Monitoring the distance through which the sealing device is propelled may be carried out either by monitoring the radio opaque front end 640 of the pusher member 608 (see FIG. 27) or by providing the outer surface of shell members 630 of the pusher member 608 with visible indications marked 669 in FIG. 27.

After completing the sealing procedure the pusher unit 608 is withdrawn and removed position of FIG. 28F) and then the retractor unit 604 may be removed from the puncture site merely by sliding it backwards in the direction of arrow 670 in FIG. 28F. Furthermore, a skilled person will appreciate that in fact, the tissue tends to act in a manner to reduce the size of the puncture, i.e. applies force in a direction to spontaneously reduce the sectional area thereof (as the direction of the force applied on the tissue-grabbing members) and thus assist in the sealing process.

Whilst some embodiments have been described and illustrated with reference to some drawings, the artisan will appreciate that many variations are

1. A sealing device for a puncture in a blood vessel, the device being slidingly receivable over a guide tube and comprising a tubular, resilient sealing member formed with a sealing portion spontaneously sealable upon deployment of the device into an activated state, an engaging portion for bearing against an external surface of the blood vessel and a plurality of anchors fitted at their fore end with fasteners for engaging a corresponding internal surface of the blood vessel and at a rear end with a manipulating bit; said anchors being displaceable between a constricted position in which they blend with the tubular sealing member and an operative position in which the fasteners are laterally expanded and engage the internal surface of the blood vessel.

2. A sealing device according to claim 1, further comprising a plurality of tissue-grabbing members adapted for engaging with tissue portion surrounding the puncture, and an arrangement for applying to said tissue-grabbing members an inwardly directed radial force.

3. A sealing device according to claim 2, wherein the fasteners are fitted with spikes facing rearwards, for engaging tissue of the blood vessel.

4. A sealing device according to claim 3, wherein at the deployed state the anchors are biased radially inwardly, for constricting the size of the puncture in the blood vessel.

5. A sealing device according to claim 1, wherein the engaging portion of the sealing member is truncated, whereby upon engaging with the blood vessel the sealing member bears at an inclination thereover.

6. A sealing device according to claim 5, wherein the fasteners are axially graded, giving rise to an imaginary path extending between edges thereof, said path conforming with the truncated edge of the sealing member.

7. A sealing device according to claim 1, wherein each anchor comprises a stem portion axially extending along the sealing member, said stem being formed at its rear end with the manipulating bit and at its fore end with the fastener.

8. A sealing device according to claim 7, wherein a first portion of the stem extends through side walls of the sealing member and a second portion of the stem extends within a common cavity of the sealing member.

9. A sealing device according to claim 8, wherein the sealing member is formed with a plurality of bores extending only a portion of the length of the sealing member and rotationally receiving the first portion of each stem.

10. A sealing device according to claim 7, wherein at an operative position of the device the sealing member applies radial force on the stems of the anchors, urging the fasteners radially inwardly for constricting the size of the puncture formed at the blood vessel.

11. A sealing device according to claim 1, wherein the anchors are formed at their aft end with a manipulating bit temporarily engageable by a deploying member for displacing into the operative position.

12. A sealing device according to claim 2, wherein the seal member is fitted with a plurality of spear members for grabbing external wall surface of the blood vessel around the puncture and constricting the size of the puncture by applying thereto force in a radially-inward direction.

13. A sealing device according to claim 12, wherein the spear members have a rear head projecting from a rear end of the sealing member, a rear stem portion axially extending through a bore of the sealing member, a fore stem portion extending through a common cavity of the sealing member and pointed tissue engaging fore ends.

14. A sealing device according to claim 1, deployable into its operate position by a deploying assembly comprising a tubular housing, a tubular controller received with the housing and a tubular pusher member received within the controller, coaxially received within one another; said pusher adapted for manipulating the sealing member into its activated position.

15. A sealing device according to claim 14, wherein the pusher member has restricted axial displacement with respect to the housing.

16. A sealing device according to claim 14, wherein the pusher member has restricted rotational displacement with respect to the housing.

17. A sealing device according to claim 14, wherein at least a fore end of the housing is radio-opaque material.

18. A sealing device according to claim 14, wherein a fore end of the housing converges to a lesser diameter, said fore end being radially expandable to admit deployment therethrough of the sealing device.

19. A sealing device according to claim 14, wherein a sleeve is fitted into the pusher via the fore end of the housing, said sleeve extending also through the sealing device and retaining it in its constricted position.

20. A sealing device according to claim 15, wherein the pusher member is formed with at least one lateral projection exiting through a corresponding slot formed in the controller and a corresponding slot formed in the housing, thereby restricting the axial displacement of the pusher within the housing.

21. A sealing device according to claim 20, wherein the at least one slot formed in the controller and in the housing are formed at their front ends with a lateral extension allowing for rotation of the pusher member about its longitudinal axis.

22. A sealing device according to claim 14, wherein the controller is axially displaceable within the housing, whereby axially displacement thereof entails intrusion of the sealing member through the fore end of the housing.

23. A sealing device according to claim 14, wherein a fore end of the pusher member is formed with activating surfaces adapted for engagement with the manipulating bits of the anchors and deploying the anchors to the operative position.

24. A sealing device according to claim 23, wherein the activating surfaces are gliding surfaces inclined so as to engage with the manipulating bits at their constricted position and where rotation of the pusher member entails sliding displacement of the manipulating bits about said activating surfaces thereby deploying the anchors into their operative position.

25. A sealing device according to claim 1, wherein the tubular sealing member is biased to axially shrink.

26. A sealing device according to claim 1, wherein the sealing portion is a diaphragm with a normally closed flow path formed by a plurality of slots, intrinsically biased into close the flow path.

27. A sealing device according to claim 1, wherein at least a component of the sealing member is made of a biodegradable or bio-absorbable material.

28. A sealing device according to claim 1, wherein the fasteners are biased for spontaneous lateral deformation wherein they are retained at their constricted position by an annular portion of the sealing member.

29. A sealing device according to claim 28, wherein a retaining member is fitted at a fore end of the sealing member for retaining the fasteners of the anchors at their constricted position.

30. A sealing device according to claim 28, wherein a rear end of the sealing member is fitted with a counter portion made of a hard material wherein a rear engaging end of the anchors bears against the counter member.

31. A sealing device according to claim 28, wherein at the constricted position the fasteners are parallel to a stem portion of the anchor and extend at all arced layout for blending with the circular pattern of the sealing member.

32. A deploying assembly for deploying a sealing device according to claim 1, comprising a tubular housing, a tubular controller received within the housing and a tabular pusher member received within the controller, coaxially received with one another, said pusher adapted for engagement with the manipulating bits of the sealing device and manipulating the sealing member into its activated position.

33. A deploying assembly for deploying a sealing device according to claim 28, comprising a tubular housing, a tubular controller coaxially received within the housing and a tabular pusher member coaxially received within the controller, said pusher adapted for engagement with a rear end of the sealing device and for manipulating it into its activated position.

34. A method for sealing a puncture in a blood vessel, the method comprising the following steps:
(i) Obtaining a sealing assembly comprising a sealing member fitted within an associated deploying assembly, with a sleeve extending through the sealing assembly, said sleeve defining a through-going path;
(ii) Introducing a medical guide tube through the path;
(iii) Removing the sleeve;
(iv) Carrying out a medical procedure through the guide tube;
(v) Slidingly displacing the sealing assembly over the guide tube until a fore end of the deploying assembly engages the blood vessel;
(vi) Expelling the sealing assembly from the deploying assembly and introducing anchor fastener members of the sealing assembly into the blood vessel through the puncture;
(vii) Deploying anchor faster members into an operative position in which they are laterally expanded and engage the internal surface of the blood vessel;
(viii) Disengaging the sealing assembly from the deploying assembly and withdrawing the guide tube, allowing a resilient sealing member formed in the sealing member to spontaneously seal.

35. A method according to claim 34, wherein at step (d) the guide tube is inserted into the blood vessel at an angle corresponding with a truncation angle of an engaging portion formed at the sealing member, for bearing against an external surface of the blood vessel.

36. A method according to claim 34, wherein at least step (e) is radio-monitored, whereby at least a fore end of the housing is radio-opaque material.

37. A method according to claim 34, wherein the anchors are biased radially inwardly, for constricting the size of the puncture in the blood vessel.

38. A method according to claim 34, wherein the fasteners of the fitted with rearwardly facing spikes whereby after step (e) a force is applied to the sealing member in a rearward direction, thereby having the spikes engage tissue of the blood vessel.

39. A method according to claim 34, wherein the deploying assembly comprises a tubular housing, a tubular controller received within the housing and a tubular pusher member received within the controller, coaxially received within one another, said pusher adapted for manipulating the sealing member into its activated position; wherein step (f) is carried out by the following steps:
(i) Axially displacing the controller with the housing in a forward direction;
(ii) Axially displacing the pusher within the controller.

40. A method according to claim 39, wherein the controller and housing are thread-engaged whereby step (a) is carried out by rotating the controller with the housing.

41. A method according to claim 39, wherein a fore end of the pusher member is formed with activating surfaces adapted for engagement with corresponding manipulating bits formed at rear ends of the anchors, whereby deploying the anchors into the operative position of step (g) is carried out by rotating the pusher within the housing.

42. A method according to claim 34. which after step (f) spear members are engaged with an external surface of the blood vessel surrounding the puncture and by applying inwardly directed radial force, the lips of the punctured blood vessel are adjoined, thereby decreasing the size of the puncture.

43. A sealing device for sealing a puncture in a blood vessel, the device is adapted for sliding over a guide tube and comprises a body member having a distal end and a proximal end; an axially resilient member extending at said distal end, with a fore end for bearing against an external surface of the blood vessel adjacent boundaries of the puncture; and an anchoring assembly at said proximal end or engaging internal wall surface of the blood vessel, said anchoring assembly comprises at least one flexible anchoring member having a constricted position for insertion into the puncture and a laterally expanded, anchoring position; and a spontaneously sealable sealing member.

44. A sealing device according to claim 43, wherein the body member is a complete enveloping wall having a cylindrical shape.

45. A sealing device according to claim 43, wherein the body member comprises a plurality of apertures.

46. A sealing device according to claim 43, wherein the body member is a cylindrical framework.

47. A sealing device according to claim 43, wherein the at least one flexible anchoring member is spontaneously deployable into its anchoring position for engagement with an inside surface of the blood vessel, adjacent boundaries of the puncture.

48. A sealing device according to claim 47, wherein the anchoring assembly comprises at least one anchoring member attached to the body member adjacent the proximal end, with a free end thereof facing the distal end of the body member, said anchoring members being elastic members normally extending in the laterally projecting position.

49. A sealing device according to claim 48, wherein the anchoring members are integrally formed with the body member or fixedly attached thereto.

50. A sealing device according to claim 43, wherein the sealing member is a membrane made of resilient material formed with a central aperture normally sealed, said aperture being a puncture rough the membrane or a plurality of slits.

51. A sealing device according to claim 43, wherein the sealing member is a resilient sleeve-like member received in the by member with a circumferential portion sealingly secured at a proximal end thereof, and flatted adjoining lips at the distal end, whereby fluid flow through the sleeve, in a direction from the distal end towards the proximal end, is not admitted.

52. A sealing device according to claim 43, wherein the resilient member as an oblique fore end for bearing against the external surface of the blood vessel adjacent boundaries of the puncture.

53. A sealing device according to claim 52, wherein the anchoring members extend at a corresponding inclination as that of the fore end of the resilient member whereby a circumferential gap between the fore end and the anchoring member is uniform.

54. A sealing device according to claim 43, wherein the anchoring assembly comprises a partitioned skirt-like portion secured at a distal end thereof to said body member and where a proximal end thereof faces the distal end of the body member.

55. A sealing device according to claim 43, wherein the anchoring assembly comprises a plurality of anchors fitted at proximal ends of axially extending studs.

56. A sealing devices according to claim 55, wherein the studs form the body member.

57. A sealing device according to claim 43, wherein the anchoring members are leaf-like members cut out from the body member where they remain attached to the body member at the proximal end.

58. A deploying assembly for introducing the sealing device according to claim 43 into the puncture vicinity, the assembly comprising a retractor unit and a pusher member, said retractor unit comprising a rigid elongate tube-embracing portion slidable over the guide tube and extending between a fore end and a rear end with an expanding member fitted at the rear end thereof for readily expanding or constricting the tube-embracing portion; said pusher member being slidable over the guide tube and within the retractor unit, with a fore end thereof engageable with the proximal end of the sealing device so as to propel it through the retractor and into the puncture of the blood vessel.

59. A deploying assembly according to claim 58, wherein the fore end of the pusher sleeve and of the retractor are radio opaque for monitoring progress of the deploying procedure.

60. A deploying assembly according to claim 58, wherein the retractor unit and the pusher member are formed with one or more axial openings to allow introducing or removal of the guide tube there through.

61. A deploying assembly according to claim 60, wherein the retractor unit and the pusher member are each formed with at least a pair of shell-like members supported at their rear end by an elastic clamp for radial displacement of said shell-like members with respect to one another, so as to facilitate mounting the retractor unit and the pusher member on the guide tube and fir expanding the retractor unit for tissue retraction.

62. A method utilizing the above different embodiment and the deploying member comprises the steps of:
(i) Obtaining a sealing assembly comprising a sealing member and an associated deploying assembly;
(ii) Introducing a medical guide tube through a path within the sealing device;
(iii) Carrying out a medical procedure through the guide tube;
(iv) Mounting a retractor of the deploying member over an exposed portion of the guide tube;
(v) Slidingly displacing the retractor over the guide tube into through tissue layers until a fore end of the retractor engages the blood vessel;
(vi) Expanding the retractor and slidingly displacing the sealing device over the guide tube and through retractor, into the vicinity of the puncture of the blood vessel;
(vii) Deploying the sealing device into its operative state within the puncture wherein the resilient member is depressed against external surface of the blood vessel and the anchoring members spontaneously deploy to their operative position in which they are laterally expanded and engage the internal space of the blood vessel;
(viii) Withdrawing the deploying member and the guide tube, allowing a resilient sealing member of the sealing member to spontaneously seal.

63. A method according to claim 62, wherein after step (c) an obturator is introduced into the guide tube for rigidifying the guide tube.

64. A method according to claim 63, wherein the obturator is formed with a radio-opaque scale to thereby facilitate monitoring the deployment process of the sealing device.
